(19) United States
(12) Patent Application Publication
(10) Pub. No.: US 2023/0251278 A1
(43) Pub. Date: Aug. 10, 2023

(54) ACQUISITION METHOD OF VALUE RELATING TO TRIGLYCERIDE METABOLIC CAPACITY, PRESENTATION METHOD OF DISEASE INFORMATION, PRESENTATION METHOD OF DISEASE DIFFERENTIATION INFORMATION, PRESENTATION METHOD OF THERAPY EFFICACY INFORMATION, PRESENTATION METHOD OF THERAPEUTIC EFFECT INFORMATION, TEST REAGENT AND TEST KIT

(71) Applicants: OSAKA UNIVERSITY, Suita-shi, Osaka (JP); RIKEN, Wako-shi, Saitama (JP); NATIONAL UNIVERSITY CORPORATION HOKKAIDO UNIVERSITY, Sapporo-shi, Hokkaido (JP)

(72) Inventors: Ken-ichi HIRANO, Suita-shi, Osaka (JP); Yasuhiro HARA, Suita-shi, Osaka (JP); Hirokazu KASHIWAGI, Suita-shi, Osaka (JP); Akira SUZUKI, Suita-shi, Osaka (JP); Takashi JIN, Wako-shi, Saitama (JP); Kenji MONDE, Sapporo-shi, Hokkaido (JP); Yuta MURAI, Sapporo-shi, Hokkaido (JP)

(21) Appl. No.: 18/014,582

(22) PCT Filed: Jul. 7, 2021
(86) PCT No.: PCT/JP2021/025687

§ 371 Date: Jan. 5, 2023

(30) Foreign Application Priority Data
Jul. 7, 2020 (JP) 2020-117350

(51) Int. Cl. G01N 33/92; G01N 33/533; C07C 229/34

(52) U.S. Cl. CPC G01N 33/92; G01N 33/533; C07C 229/34; G01N 2800/323; G01N 2800/7085; G01N 2800/325

(57) ABSTRACT

Provided are a method for acquiring a value relating to a triglyceride metabolic capacity of a subject within a shorter period of time compared to conventional nuclear medicine methods, and a test reagent and a test kit that are to be used in the acquisition method. This problem can be solved by an acquisition method of a value relating to the triglyceride metabolic capacity in leucocytes of a subject, said method comprising: a first step of mixing, in vitro, a fatty acid compound labeled with a fluorescent substance with leucocytes collected from the subject and thus bringing the fatty acid labeled with the fluorescent substance into contact with the leucocytes, wherein the fatty acid compound contains a fatty acid residue, the fatty acid residue has 8-26 carbon atoms, and a part of hydrogen atoms constituting the fatty acid residue, excluding a terminal methyl group of the fatty acid residue, may be substituted by an alkyl group having 1-3 carbon atoms; and a second step of acquiring, as the value relating to the triglyceride metabolic capacity, a measured fluorescence intensity that is derived from the fluorescent label in the leucocytes.

### TECHNICAL FIELD

Disclosed herein are a method for acquiring a value relating to a triglyceride metabolic capacity, a method for presenting disease information, a method for presenting disease differentiation information, a method for presenting therapy efficacy information, a method for presenting therapeutic effect information, a test reagent, and a test kit.

### BACKGROUND ART

Triglyceride deposit cardiomyovasculopathy (TGCV) is a novel disease entity found from cardiac transplantation cases in Japan in 2008. TGCV is an orphan disease that results in severe heart failure, arrhythmia, and coronary artery disease as a result of accumulation of triglyceride in heart muscle and a coronary artery. It is estimated that there are 40 to 50 thousand potential patients with TGCV in the whole country, and that a TGCV preliminary group has several tens of thousands of people. However, the number of actual cases of TGCV diagnosed remains 226 (of which 46 were dead) at the time point of November 2019 (Non Patent Literatures 1 to 6).

### CITATION LIST

### Non Patent Literature

Non Patent Literature 1: Li M, et. al. Orphanet J Rare Dis 2019; 14:134. doi:10.1186/s13023-019-1087-4.
Non Patent Literature 2: Miyauchi H, et. al. Ann Nucl Cardiol 2018; 4:94-100.
Non Patent Literature 3: Higashi M, et. al. Ann Nucl Cardiol 2017; 3:94-102.
Non Patent Literature 4: Hirano K, et. al. Eur Heart J 2015; 36:580.
Non Patent Literature 5: Ikeda Y, et. al. Eur Heart J 2014; 35:875.
Non Patent Literature 6: Hirano K, et. al. N Engl J Med 2008; 359:2396-2398.

### SUMMARY OF INVENTION

### Technical Problem

As the reason why the number of actual cases of TGCV diagnosed and the number of potential patients with TGCV greatly deviate from each other, it is included that the diagnosis of TGCV is currently performed by nuclear medicine methods using a ¹²³I-labeled β-methyl iodophenyl-pentadeca-noic acid (¹²³I-BMIPP) scintigram. That is, the ¹²³I-BMIPP (trade name: CARDIODINE® Injection) is administered to a patient, the heart is imaged with SPECT 30 minutes later, a redistribution image of the ¹²³I-BMIPP of the heart is taken 3 to 4 hours later, and a triglyceride metabolic capacity of the heart is evaluated. The ¹²³I-BMIPP scintigram is performed in vivo, and thus patients are needed to be constrained for an extended period of time. In addition, there are few medical facilities that own the nuclear medicine laboratory itself, and in Japan, there are only a little over 10 facilities that can perform the scintigram. It can be considered that the inspection for diagnosing TGCV cannot be easily performed from such an inspection environment, and the number of inspections itself is reduced.

An object of the present invention is to provide a method for acquiring a value relating to a triglyceride metabolic capacity of a subject within a shorter time compared to conventional nuclear medicine methods, and a test reagent and a test kit that are to be used in the acquisition method.

### Solution to Problem

As a result of diligent research, the present inventors found that the use of a fluorescence-labeled fatty acid can acquire the value relating to the triglyceride metabolic capacity of the subject in a shorter time compared to the conventional nuclear medicine methods.

The present invention has been achieved based on the above findings, and includes following aspects.

### Item 1.

A method for acquiring a value relating to a triglyceride metabolic capacity in leucocytes of a subject, comprising following steps of:

a first step of mixing, in vitro, a fatty acid compound labeled with a fluorescent substance with leucocytes collected from the subject and thus bringing the fatty acid labeled with the fluorescent substance into contact with the leucocytes, wherein the fatty acid compound contains a fatty acid residue, the fatty acid residue has 8 to 26 carbon atoms, and a part of hydrogen atoms constituting the fatty acid residue, excluding a terminal methyl group of the fatty acid residue, may be substituted by an alkyl group having 1 to 3 carbon atoms; and

a second step of acquiring, as the value relating to the triglyceride metabolic capacity, a measured fluorescence intensity that is derived from a fluorescent label in the leucocytes.

### Item 2.

The method according to item 1, wherein a hydrogen atom of the terminal methyl group of the fatty acid residue is substituted with a substituted or unsubstituted phenyl group.

### Item 3.

The method according to item 1, wherein the fatty acid compound is represented by the following general formula 1:

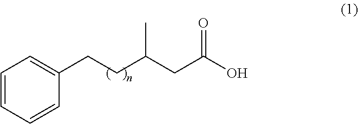

(n is an integer of 4 to 22).

### Item 4.

The method according to item 1, wherein the fatty acid compound labeled with the fluorescent substance is represented by the following general formula 2:

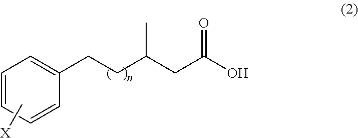

(n is the integer of 4 to 22. X includes the fluorescent substance and a linker moiety).

### Item 5.

The method according to any one of items 1 to 4, further comprising

a third step of removing the fluorescence-labeled fatty acid compound from the leucocytes over a period of 5 minutes to 2 hours after the second step;

a fourth step of acquiring a measured fluorescence intensity that is derived from the fluorescent label in the leucocytes after the third step; and

a fifth step of obtaining a value indicating a difference between a first measurement value and a second measurement value using the measured fluorescence intensity acquired in the second step as the first measurement value and the measured fluorescence intensity acquired in the fourth step as the second measurement value, and acquiring the value indicating the difference as the value relating to the triglyceride metabolic capacity.

### Item 6.

A method for presenting disease information, comprising

comparing the value relating to the triglyceride metabolic capacity in the leucocytes of the subject acquired by the method for acquiring the value according to any one of items 1 to 5 with a predetermined reference range of the triglyceride metabolic capacity, and suggesting that the subject is triglyceride deposit cardiomyovasculopathy, triglyceride deposit-type atherosclerosis, or a preliminary group of these diseases when the triglyceride metabolic capacity in the leucocytes of the subject is out of the reference range.

### Item 7.

A method for presenting disease differentiation information, comprising comparing the value relating to the triglyceride metabolic capacity in the leucocytes of the subject acquired by the method for acquiring the value according to any one of items 1 to 5 with the predetermined reference range of the triglyceride metabolic capacity, and suggesting that the subject is cholesterol deposit-type atherosclerosis or a preliminary group thereof when the triglyceride metabolic capacity in the leucocytes of the subject is within the reference range.

### Item 8.

A method for presenting disease differentiation information, comprising comparing the value relating to the triglyceride metabolic capacity in the leucocytes of the subject acquired by the method for acquiring the value according to any one of items 1 to 5 with the predetermined reference range of the triglyceride metabolic capacity, and suggesting that the subject is not dilated cardiomyopathy when the triglyceride metabolic capacity in the leucocytes of the subject is out of the reference range.

### Item 9.

A method for presenting efficacy information on a vasodilation therapy using a stent, comprising

comparing the value relating to the triglyceride metabolic capacity in the leucocytes of the subject acquired by the method for acquiring the value according to any one of items 1 to 5 with the predetermined reference range of the triglyceride metabolic capacity, and suggesting that the vasodilation therapy using the stent is not effective in the subject when the triglyceride metabolic capacity in the leucocytes of the subject is out of the reference range.

### Item 10.

A method for presenting efficacy information of a composition, comprising

comparing the value relating to the triglyceride metabolic capacity in the leucocytes of the subject acquired by the method for acquiring a value according to any one of items 1 to 5 with the predetermined reference range of the triglyceride metabolic capacity, and suggesting that administration of a medium chain fatty acid composition and/or administration of a composition for regression of triglyceride deposit-type atherosclerosis to the subject is effective when the triglyceride metabolic capacity in the leucocytes of the subject is out of the reference range.

### Item 11.

A method for presenting therapeutic effect information, comprising

comparing the value relating to the triglyceride metabolic capacity in the leucocytes of the subject acquired by the method for acquiring the value according to any one of items 1 to 5 with the value relating to the triglyceride metabolic capacity of the same subject in the past, and suggesting that a therapy applied to the subject is effective when the value relating to the triglyceride metabolic capacity in the leucocytes of the subject is higher than the value relating to the triglyceride metabolic capacity of the same subject in the past.

### Item 12.

A computing device comprising a processing unit that executes each of the steps according to item 8, 9, 10, or 12.

### Item 13.

A computer program that, when executed by a computer, causes the computing device to execute each of the steps according to item 8, 9, 10, or 12.

### Item 14.

A blood collection tube used for blood collection for collecting leucocytes of a subject, wherein

the leucocytes are used to acquire the value relating to the triglyceride metabolic capacity according to any one of items 1 to 5 in vitro, and

the blood collection tube contains a specific gravity liquid for separating the leucocytes, or the specific gravity liquid for separating the leucocytes and a leucocyte separating agent.

### Item 15.

A test reagent for evaluating a triglyceride metabolic capacity, comprising a fatty acid compound labeled with a fluorescent substance, wherein a fatty acid compound contains the fatty acid residue, the fatty acid residue has 8 to 26 carbon atoms, and a part of a hydrogen atoms constituting the fatty acid residue, excluding the terminal methyl group of the fatty acid residue, may be substituted with an alkyl group having 1 to 3 carbon atoms.

### Item 16.

A test kit comprising the blood collection tube according to item 14 and the test reagent according to item 15 in combination.

### Item 17.

A compound represented by the following general formula 3:

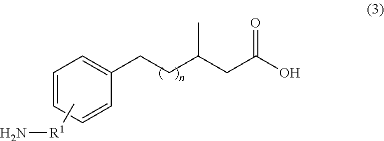

(n is the integer of 4 to 22. R¹ represents an alkylene group having 3 to 6 carbon atoms).

### Item 18.

A method for producing the compound represented by the following general formula 3:

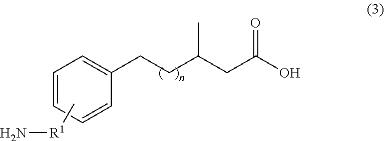

(n is an integer of 4 to 22. R¹ represents an alkylene group having 3 to 6 carbon atoms.) comprising

removing a tert-butoxycarbonyl protecting group from a compound represented by the following general formula 4:

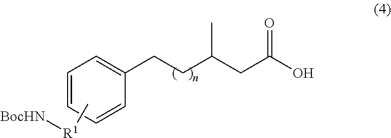

(n is the integer of 4 to 22. R¹ represents the alkylene group having 3 to 6 carbon atoms. Boc represents the tert-butoxycarbonyl protecting group).

### Item 19.

A compound represented by the following general formula 2:

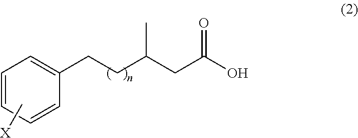

(n is the integer of 4 to 22. X includes the fluorescent substance and the linker moiety).

### Item 20.

A method for producing the compound represented by the following general formula 2;

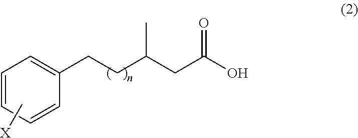

(n is the integer of 4 to 22. X includes the fluorescent substance and the linker moiety.) comprising

binding the compound represented by the following general formula 3:

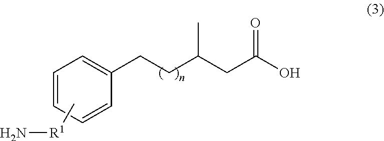

formula 3
(n is the integer of 4 to 22. R¹ represents the alkylene group having 3 to 6 carbon atoms.) to the fluorescent substance by amine coupling.

### Advantageous Effects of Invention

According to the present invention, the triglyceride metabolic capacity can be evaluated more easily compared to a method using conventional nuclear medicine methods. In addition, according to the method for evaluating the triglyceride metabolic capacity according to the present invention, disease information, disease differentiation information, disease therapy information, and the like can be presented for a patient who has undergone an inspection.

### BRIEF DESCRIPTION OF DRAWINGS

FIG. 1 shows a summary of a method for acquiring a value relating to a triglyceride metabolic capacity. FIG. 1(A) shows an outline of metabolism of fluorescence-labeled fatty acid compounds in cells. FIG. 1(B) shows one embodiment of the acquisition method. FIG. 1(C) shows another embodiment of the acquisition method.

FIG. 2 shows a synthesis scheme of the fatty acid compound. FIG. 2(A) shows synthesis step Ia. FIG. 2(B) shows synthesis step Ib. FIG. 2(C) shows synthesis step II. FIG. 2(D) shows synthesis step III. FIG. 2(E) shows synthesis step IV.

FIG. 3 shows an example of an outline drawing of presentation system 1000, 2000, 3000, 4000, or 5000.

FIG. 4 shows an example of a hardware configuration of presentation device 10, 20, 30, 40, or 50.

FIG. 5 shows functional blocks of the presentation device 10, 20, 30, 40, or 50.

FIG. 6 shows a flow of a processing of disease information presentation program.

FIG. 7 shows a flow of a processing of a presentation program for differentiation information of cholesterol deposit-type atherosclerosis or a preliminary group thereof.

FIG. 8 shows a flow of a processing of a presentation program for differentiation information of dilated cardiomyopathy.

FIG. 9 shows a flow of a processing of a presentation program for efficacy information on a vasodilation therapy using a stent.

FIG. 10 shows a flow of a processing of a presentation program for efficacy information on a medium chain fatty acid composition and/or a composition for regression of triglyceride deposit-type atherosclerosis.

FIG. 11 shows a flow of a processing of a presentation program for therapeutic effect information.

FIG. 12 shows a flowchart for outputting information indicating a difference between a first measurement value and a second measurement value.

FIG. 13 shows ability for uptake of Alexa Fluor™ 680-BMPP.

FIG. 14 shows ability for uptake of a fluorescein-labeled BMPP.

FIG. 15 shows ability of leucocytes to excrete the Alexa Fluor™ 680-BMPP in healthy individuals and patients with primary triglyceride deposit cardiomyovasculopathy.

FIG. 16 shows ability of leucocytes to excrete the Alexa Fluor™ 680 in the healthy individuals and the patients with primary triglyceride deposit cardiomyovasculopathy.

FIG. 17 shows results of an examination of amount of the Alexa Fluor™ 680-BMPP excreted based on an excretion time.

FIG. 18 shows results of administrating medium chain fatty acid to rats. FIG. 18(A) shows incidence of abdominal aortic aneurysm. FIG. 18(B) shows presence or absence of an occurrence of rupture of aortic aneurysm.

FIG. 19 shows cross-sectional views of a blood vessel showing a result of performing MCT dietary therapy for 50 days on patients in case 1 and evaluating lipid accumulation in coronary arteries before and after the therapy. FIG. 19(A) shows the result before the start of the MCT dietary therapy. FIG. 19(B) shows the result after 50 days from the start of the MCT dietary therapy.

FIG. 20 shows diagrams showing results (distance from the coronary artery ostium and diameter of blood vessel) of performing MCT dietary therapy for 4 years on patients in case 2 and evaluating lipid accumulation in the coronary artery before and after the therapy. FIG. 20(A) shows the result before the start of the MCT dietary therapy. FIG. 20(B) shows the result after 4 years from the start of the MCT dietary therapy.

FIG. 21 shows cross-sectional views of a blood vessel showing the result of performing the MCT dietary therapy for 4 years on the patients in the case 2 and evaluating lipid accumulation in the coronary arteries before and after the therapy. FIG. 21(A) shows the result before the start of the MCT dietary therapy. FIG. 21(B) shows the result after 4 years from the start of the MCT dietary therapy.

FIG. 22(A) shows, in the upper part, ability of the leucocytes to excrete the Alexa Fluor™ 680-BMPP in the patients before the start of the therapy, and in the lower part, ability of the leucocytes to excrete the Alexa Fluor™ 680-BMPP in the healthy individuals. FIG. 22(B) shows, in the upper part, ability of the leucocytes to excrete the Alexa Fluor™ 680-BMPP in the patients after the therapy, and in the lower part, ability of the leucocytes to excrete the Alexa Fluor™ 680-BMPP in the healthy individuals.

### DESCRIPTION OF EMBODIMENTS

### 1. Method for Acquiring Value Relating to Triglyceride Metabolic Capacity

An embodiment of the present invention relates to a method for acquiring a value relating to a triglyceride metabolic capacity in a cell of a subject. The acquisition method includes a first step of mixing, in vitro, a fatty acid compound labeled with a fluorescent substance with leucocytes collected from the subject and thus bringing the fatty acid labeled with the fluorescent substance into contact with the leucocytes, and a second step of acquiring a measured fluorescence intensity that is derived from the fluorescent label in the leucocytes. The fatty acid compound labeled with the fluorescent substance is also referred to as a “fluorescence-labeled fatty acid compound” in the present description. The method for acquiring the value relating to the triglyceride metabolic capacity is hereinafter sometimes simply referred to as “acquisition method”.

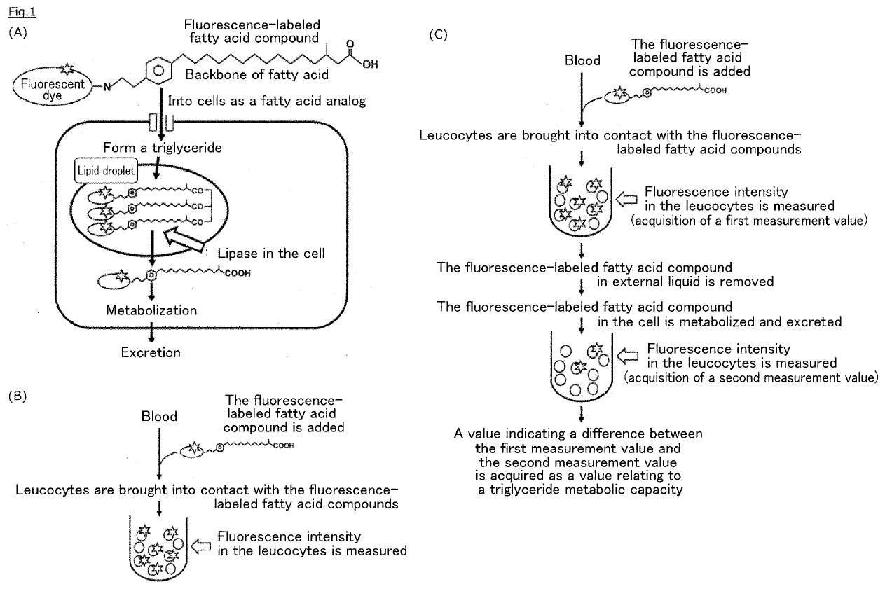

The method of acquiring the value relating to the triglyceride metabolic capacity will be described with reference to FIG. 1. FIG. 1(A) shows a scheme of uptake of the fluorescence-labeled fatty acid compounds into the cells and excretion of the fluorescence-labeled fatty acid compounds from the cells dependent on lipase activity. The fluorescence-labeled fatty acid compound is taken into the cell as a fatty acid analog and esterified into a trimer to form a triglyceride. The triglyceride is rapidly metabolized by action of the lipase in the cell, and is converted again into monomeric fatty acid analog, which is then excreted into the cell.

FIG. 1(B) is a scheme illustrating an example of the acquisition method including a first step and a second step. In the first step, the blood sample collected from the subject and the fluorescence-labeled fatty acid compounds are mixed in vitro, so that the leucocytes in the blood sample are brought into contact with the fluorescence-labeled fatty acid compounds.

Next, in the second step, fluorescence intensity of the fluorescence-labeled fatty acid compound taken in the leucocytes is measured, and the measured value is acquired as the value relating to the triglyceride metabolic capacity.

FIG. 1(C) includes a third step, a fourth step, and a fifth step in addition to the first step and the second step. In the third step, external liquid in which the cells and the fluorescence-labeled fatty acid compounds are suspended is removed after the second step. Thereby, fluorescence-labeled fatty acid compounds excreted from the leucocytes are also removed.

In the fourth step, a measured fluorescence intensity that is derived from the fluorescent substance bonded to the fluorescence-labeled fatty acid compound in the leucocytes is acquired after the third step.

In the fifth step, a value indicating a difference between a first measurement value and a second measurement value is obtained using the measured fluorescence intensity acquired in the second step as the first measurement value and the measured fluorescence intensity acquired in the fourth step as the second measurement value. Then, the value indicating the difference is acquired as the value relating to the triglyceride metabolic capacity.

In the acquisition method, the subject is not limited as long as the subject is a subject to be inspected for the triglyceride metabolic capacity. For example, patients with triglyceride deposit cardiomyovasculopathy or triglyceride deposit-type atherosclerosis, or a preliminary group thereof may have normal triglyceride levels in serum in some cases. Therefore, a person who is not diagnosed to have a disorder of lipid metabolism by a normal blood test can be the subject. Herein, the “preliminary group” of the disease means a person who has not developed the disease to be inspected at an inspection stage but may develop the disease to be inspected at the inspection stage in the future.

The leucocytes collected from the subject are not limited as long as they survive to such an extent that the bioactivity of the cells can be measured. The blood collection for collecting leucocytes is not limited as long as the blood collection is performed using an anticoagulant. As the anticoagulant, heparin salt, Ethylenediaminetetraacetic acid (EDTA) salt, sodium citrate, CPD solution, ACD solution, and the like can be used. The leucocytes to be brought into contact with the fluorescence-labeled fatty acid compound may be in a state of being contained in whole blood or diluted blood obtained by diluting whole blood. For diluting whole blood, saline, cell culture medium, specific gravity liquid for leucocyte separation described later, and the like can be used. The leucocytes to be brought into contact with the fluorescence-labeled fatty acid compound may be leucocyte fraction obtained by removing erythrocyte fraction from whole blood. In this case, platelets may be contained or may not be contained.

The type of leucocyte contained in the leucocyte fraction is not limited. A neutrophil, a lymphocyte, a monocyte, an eosinophil, and a basophil may be all used. Preferably, the neutrophil and the lymphocyte can be used. Hereinafter, a sample containing the leucocyte for measurement is referred to as a leucocyte suspension. The liquid in which the cells are suspended is referred to as external liquid.

When removing erythrocytes, it is preferable not to use a hemolytic agent. When the hemolytic agent is used, it is preferable to hemolyze the erythrocytes immediately before the measurement of the fluorescence intensity. This is to prevent leucocytes from being killed by the hemolytic agent. Therefore, it is preferable to remove erythrocytes using a specific gravity liquid for leucocyte separation such as HetaSep (Beritas Corporation) or Lymphocyte™-poly Cell Separation Media (having 1.113±0.001 g/cm³ density at 22° C.), or a thixotropic gel described in U.S. Pat. No. 5,667,963A or the like. In addition, in a case where the hemolytic agent is used, for example, BD FACS Lysing Solution (BD Biosciences) or the like can be used.

The contact between the fluorescence-labeled fatty acid compound and the leucocytes in the first step is not limited as long as the contact is performed under a condition that the fluorescence-labeled fatty acid compound can be incorporated in a state where the leucocyte survives. Details of the fluorescence-labeled fatty acid compound are described below.

The contact between the fluorescence-labeled fatty acid compound and the leucocytes can be performed, for example, by adding the fluorescence-labeled fatty acid compound to the leucocyte suspension or by mixing a solution of the fluorescence-labeled fatty acid compound and the leucocyte suspension to prepare a mixture of the fluorescence-labeled fatty acid compound and the leucocytes. A concentration of the fluorescence-labeled fatty acid compound to be added to the leucocyte suspension is not limited as long as the concentration is a concentration at which the leucocyte is not killed and is a concentration at which the fluorescent substance in the leucocyte can be measured by a measurement instrument described later. For example, a final concentration of the fluorescence-labeled fatty acid compound in the mixture of the fluorescence-labeled fatty acid compound and the leucocytes can be about 0.005 μM to 1 mM. If the fluorescent substance is, for example, a fluorescent substance of Alexa™ series or a fluorescent substance having fluorescence intensity which is equal to or higher than that of equivalent thereof, the final concentration of the fluorescence-labeled fatty acid compound can be about 0.005 μM to 0.5 μM. If the fluorescent substance is, for example, a fluorescent substance such as FITC or rhodamine or a fluorescent substance having equivalent fluorescence intensity thereof, the final concentration of the fluorescence-labeled fatty acid compound can be about 0.1 μM to 1 mM. The number of the leucocytes contained in the mixture of the fluorescence-labeled fatty acid compound and the leucocytes can be about 103 to 107.

A contact time between the fluorescence-labeled fatty acid compound and the leucocytes can be, for example, 10 minutes to 3 hours, preferably 30 minutes to 1 hour. A temperature at which the fluorescence-labeled fatty acid compound and the leucocytes are brought into contact with each other can be about 20° C. to 30° C., preferably about 23° C. to 28° C.

In the second step, the fluorescence intensity of the fluorescent substance bound to the fluorescence-labeled fatty acid compound in the leucocyte is measured. The fluorescence intensity of the fluorescent substance in the leucocyte can be measured using, for example, a flow cytometer. In this case, the external liquid containing the fluorescence-labeled fatty acid compound that is not taken in the leucocyte may or may not be removed. For example, as another method, the leucocytes may be collected from the mixture of the fluorescence-labeled fatty acid compound and the leucocytes by centrifugation or filtering to measure the fluorescence intensity of the fluorescent substance in the leucocyte using a fluorometer or the like. In this case, the leucocytes may or may not be lysed. However, the measurement of a case in which the leucocytes are lysed is more accurate.

When the third step is carried out, the leucocytes are collected from the mixture of the fluorescence-labeled fatty acid compound and the leucocytes according to the method exemplified in the second step, and the external liquid is replaced with a new external liquid such as a saline containing no fluorescence-labeled fatty acid compound and a cell culture medium. It is preferable that the external liquid does not allow the leucocytes to excrete the fluorescence-labeled fatty acid compound by its own action. Therefore, it is preferable that the external liquid is an aqueous solution which does not damage a cell membrane or change osmotic pressure in a cell. After the external liquid is replaced, the mixture is left to stand for 5 minutes to 2 hours, preferably 10 minutes to 30 minutes. By this standing, the monomer fluorescence-labeled fatty acid compound is excreted from the leucocytes.

The fourth step after the third step is the same as the second step.

In the fifth step, the value indicating the difference between the first measurement value and the second measurement value is obtained using the measured fluorescence intensity acquired in the second step as the first measurement value and the measured fluorescence intensity acquired in the fourth step as the second measurement value. The first measurement value acquired in the second step is a measurement value in a situation that the fluorescence-labeled fatty acid compound is taken into and stored in the leucocyte. The second measurement value acquired in the fourth step is a measurement value in a situation that the fluorescence-labeled fatty acid compound which had been taken into the leucocyte has been excreted from the leucocyte. Therefore, the difference between the first measurement value and the second measurement value become large in a subject having high triglyceride metabolic capacity. On the other hand, in a subject having low triglyceride metabolic capacity, the difference between the first measurement value and the second measurement value become small.

Here, in a case where the fluorescence intensity of the leucocytes is measured using the flow cytometer, for example, the fluorescence intensity can be represented by a histogram expressed by two axes of the fluorescence intensity of each leucocyte and the number of leucocytes distributed within a range of a predetermined fluorescence intensity value. The first measurement value and the second measurement value of the fluorescence intensity can be expressed as a mode (i.e., peak value) of fluorescence intensity in the histogram acquired by the flow cytometer. Alternatively, the fluorescence intensity value can be expressed as the number of cells in each fluorescence intensity of the histogram acquired by the flow cytometer or the distribution range of the fluorescence intensity value of the histogram. The fluorescence intensity value obtained from the histogram obtained from the flow cytometer may be the value as it is, or may be subjected to normalization, conversion to a relative value, or the like.

When the fluorescence intensity of the leucocytes is measured using the fluorometer, for example, the fluorescence intensity can be expressed as the fluorescence intensity of the sample containing the leucocytes. At this time, it is preferable to make the number of leucocytes equal among the samples, or to correct the fluorescence intensity based on the number of leucocytes after the measurement.

When the fluorescence intensity of the leucocytes is measured using the fluorometer, for example, the fluorescence intensity is preferably expressed as the fluorescence intensity of a sample containing the fluorescence-labeled fatty acid compound released from the sample in which the leucocytes are lysed. In this case, when preparing the sample in which the leucocyte is lysed, it is preferable to make the number of leucocytes before lysing equal among the samples, or to correct, after the measurement, the fluorescence intensity based on the number of leucocytes before lysing. The fluorescence intensity value obtained from the flow cytometer may be the value as it is, or may be subjected to normalization, conversion to a relative value, or the like.

When the fluorescence intensity of the leucocytes is measured using the fluorometer, for example, the fluorescence intensity can be expressed as the fluorescence intensity of the leucocytes, without the leucocytes being lysed, in which the fluorescence-labeled fatty acid compound is taken. For example, in the first step, the blood sample collected from the subject is brought into contact with a solid phase in vitro, thereby binding the leucocytes in the blood sample to the solid phase. The solid phase is not particularly limited in shape, and includes, for example, a microplate, a microtube, a test tube, a bead, and a membrane. The material of the solid phase is not particularly limited, and for example, polystyrene, polypropylene, or the like can be used for the microplate, the microtube, the test tube, or the like. For the bead, polystyrene Xmap™ beads (Luminex Corporation), MagPlex® microspheres (Luminex Corporation), or the like can be used. For the membrane, a nitrocellulose filter, a nylon filter, or the like can be used. The leucocyte may be bound to the solid phase via, for example, a capture antibody capable of capturing a leucocyte. The capture antibody is not limited as long as it can capture a leucocyte. The capture antibody may be immobilized on the solid phase in advance, or may be immobilized on the solid phase after the leucocyte and the capture antibody are bound to each other.

A method for immobilizing the capture antibody on the solid phase is known. In addition, the capture antibody is known. For example, as a carrier on which the capture antibody is immobilized in advance, Dynabeads series manufactured by Thermo Fisher Scientific Inc. using magnetic beads can be exemplified.

The contact between the fluorescence-labeled fatty acid compound and the leucocyte may be performed after the leucocyte is bound to the solid phase. Alternatively, the leucocytes in which the fluorescent-labeled fatty acid compound is taken may be bound to the solid phase after the fluorescent-labeled fatty acid compound and the leucocyte are brought into contact with each other. The former is preferable.

After the first step and before the second step, an operation of washing the solid phase for removing cells or antibodies that are not bound to the solid phase may be comprised. When the solid phase is washed, for example, the external liquid such as the saline containing no fluorescence-labeled fatty acid compound and the cell culture medium used in the third step can be used.

In the second step, the fluorescence intensity of the cell bound to the solid phase is measured. Further, the third step and the fourth step are also carried out in a state where the leucocyte is bound to the solid phase. The fluorescence intensity value obtained from the fluorometer may be the value as it is, or may be subjected to normalization, conversion to a relative value, or the like.

The value indicating the difference between the first measurement value and second measurement value of the fluorescence intensity can be used as the value relating to the triglyceride metabolic capacity. Here, the value indicating the difference is not limited as long as the difference between the first measurement value and the second measurement value is shown. For example, the value indicating the difference may be a subtraction value obtained by subtracting the second measurement value from the first measurement value; a division value obtained by dividing the first measurement value by the second measurement value or a value obtained by converting the division value into a percentage; a relative value of one value with respect to the other value; a subtraction value obtained by subtracting the first measurement value from the second measurement value; a division value obtained by dividing the second measurement value by the first measurement value or a value obtained by converting the division value into a percentage, or the like. When the value indicating the difference is the subtraction value obtained by subtracting the second measurement value from the first measurement value; or, the division value obtained by dividing the first measurement value by the second measurement value or the value obtained by converting the division value into the percentage, the larger the difference between the first measurement value and the second measurement value, the larger the value indicating the difference. When the value indicating the difference is the subtraction value obtained by subtracting the first measurement value from the second measurement value; or, the division value obtained by dividing the second measurement value by the first measurement value or the value obtained by converting the division value into the percentage, the larger the difference between the first measurement value and the second measurement value, the smaller the value indicating the difference.

Furthermore, the acquisition method may comprise a sixth step of determining whether or not the value relating to the triglyceride metabolic capacity of the triglycerides acquired in the second step or the value relating to the triglyceride metabolic capacity acquired in the fifth step is within a predetermined reference range.

When it is determined whether or not the value relating to the triglyceride metabolic capacity of the triglycerides acquired in the second step or the sixth step is within the predetermined reference range, if the value relating to the triglyceride metabolic capacity of the subject is equivalent to, for example, the value relating to the triglyceride metabolic capacity of a healthy subject, it can be determined that the value relating to the triglyceride metabolic capacity of the subject is within the reference range. The predetermined reference range can be a range obtained by adding a certain width to a reference value. The reference value can also be determined by receiver operating characteristic (ROC) curve, a discriminant analysis method, a mode method, a Kittler method, a three-sigma method, a percentile method, or the like. In addition, sensitivity, specificity, negative predictive value, positive predictive value, and first quartile can be exemplified as the reference value. The certain width added to the reference value can be determined by standard deviation (SD), or variance (CV). For example, the certain width added to the reference value may be ±2SD, ±1SD, or ±CV. When the value indicating the difference is the subtraction value obtained by subtracting the second measurement value from the first measurement value; or, the division value obtained by dividing the first measurement value by the second measurement value or the value obtained by converting the division value into the percentage, if the value relating to the triglyceride metabolic capacity of the subject is lower than the reference range, it can be determined that the value is out of the reference range. When the value indicating the difference is the subtraction value obtained by subtracting the first measurement value from the second measurement value; or, the division value obtained by dividing the second measurement value by the first measurement value or the value obtained by converting the division value into the percentage, if the value relating to the triglyceride metabolic capacity of the subject is higher than the reference range, it can be determined that the value is out of the reference range.

In addition, when the value relating to the triglyceride metabolic capacity of the subject is from more than 0.9 times to less than 1.1 times as large as the value relating to the triglyceride metabolic capacity of the healthy subject, it can be determined that the value relating to the triglyceride metabolic capacity of the subject is equivalent to the value relating to the triglyceride metabolic capacity of the healthy subject.

When the value indicating the difference is the subtraction value obtained by subtracting the second measurement value from the first measurement value; or, the division value obtained by dividing the first measurement value by the second measurement value or the value obtained by converting the division value into the percentage, if the value relating to the triglyceride metabolic capacity of the subject is 0.9 times or less, preferably 0.7 times or less, more preferably 0.5 times or less as large as the value of the value relating to the triglyceride metabolic capacity of the healthy subject, it can be determined that the triglyceride metabolic capacity of the subject is out of the reference range. Furthermore, when the value relating to the triglyceride metabolic capacity of the subject is out of the reference range, it can be determined that the triglyceride metabolic capacity of the subject is low, or it can be suggested that the triglyceride metabolic capacity of the subject is low.

When the value indicating the difference is the subtraction value obtained by subtracting the first measurement value from the second measurement value; or, the division value obtained by dividing the second measurement value by the first measurement value or the value obtained by converting the division value into the percentage, if the value relating to the triglyceride metabolic capacity of the subject is 1.1 times or more, preferably 1.3 times or more, more preferably 1.5 times or more as large as the value relating to the triglyceride metabolic capacity of the healthy subject, it can be determined that the triglyceride metabolic capacity of the subject is out of the reference range. Furthermore, when the value relating to the triglyceride metabolic capacity of the subject is out of the reference range, it can be determined that the triglyceride metabolic capacity of the subject is low, or it can be suggested that the triglyceride metabolic capacity of the subject is low.

### 2. Presentation Method of Information

In the present description, the method for presenting information using the value relating to the triglyceride metabolic capacity acquired in the above 1. More specifically, when the triglyceride metabolic capacity in the leucocytes of the subject is out of the predetermined reference range of the triglyceride metabolic capacity, a predetermined information is presented. Here, in each of the presentation methods described below, the “suggesting” may include indicating a possibility and indicating a disease. Preferably, the “suggesting” is indicating a possibility. The “presenting” may include displaying related information on a display unit of a personal computer or a tablet used in a hospital, or printing the information on a paper medium so that a doctor, a medical technologist, a nurse, or the like can recognize the related information.

### 2-1. Presentation Method of Disease Information

A method for presenting disease information comprises a step of suggesting that, when the value relating to the triglyceride metabolic capacity in leucocytes of the subject acquired according to the acquisition method described in the above 1. is compared with the predetermined reference range of the triglyceride metabolic capacity, if the triglyceride metabolic capacity in leucocytes of the subject is out of the reference range, the subject is the triglyceride deposit cardiomyovasculopathy, the triglyceride deposit-type atherosclerosis, or the preliminary group of these diseases. The triglyceride deposit cardiomyovasculopathy is a state in which the triglyceride metabolic capacity in cells is reduced and triglycerides are accumulated in blood vessel cells of blood vessels in heart muscle. The triglyceride deposit-type atherosclerosis is a state in which triglycerides accumulate in cells constituting arteries and arteriosclerosis occurs. The triglyceride deposit-type atherosclerosis causes diffuse hardening in blood vessels, unlike cholesterol deposit-type arteriosclerosis caused by localized vascular deposition of cholesterol. For the cholesterol deposit-type atherosclerosis, preliminary group thereof can be predicted by total cholesterol levels, HDL-cholesterol levels, and LDL-cholesterol levels in blood. However, the onset of the triglyceride deposit cardiomyovasculopathy and the triglyceride deposit-type arteriosclerosis does not necessarily correlate with triglyceride levels in blood, and thus the present method is useful. In addition, since the present method can predict the possibility of accumulation of triglycerides in vascular endothelial cells using peripheral leucocytes, the test is convenient and can be performed in a short time.

### 2-2. Presentation Method of Differentiation Information of Cholesterol Deposit-Type Atherosclerosis or Preliminary Group Thereof

A presentation method of differentiation information of the cholesterol deposit-type atherosclerosis or the preliminary group thereof according to the present embodiment comprises a step of comparing the value relating to the triglyceride metabolic capacity in leucocytes of the subject acquired according to the acquisition method described in the above 1. with the predetermined reference range of the triglyceride metabolic capacity, and suggesting that, if the triglyceride metabolic capacity in leucocytes of the subject is within the reference range, the subject is the cholesterol deposit-type atherosclerosis or the preliminary group thereof. The suggesting step may be a step of comparing value relating to the triglyceride metabolic capacity in the leucocytes of the subject with the predetermined reference range of the triglyceride metabolic capacity, and suggesting that, if the triglyceride metabolic capacity in the leucocytes of the subject is out of the reference range, the subject is not the cholesterol deposit-type atherosclerosis or the preliminary group thereof. Alternatively, the suggesting step may be a step of suggesting whether or not the subject is the cholesterol deposit-type atherosclerosis or the preliminary group thereof depending on whether the triglyceride metabolic capacity in the leucocytes of the subject is within or out of the reference range.

In the aspect of the present embodiment, being opposite to the aspect described in the above 2-1, information for differentiating, for example, whether the subject is the cholesterol deposit-type atherosclerosis or the triglyceride deposit-type atherosclerosis, or whether the subject is the preliminary group of the cholesterol deposit-type atherosclerosis or the preliminary group of the triglyceride deposit-type atherosclerosis is presented. A local stenting can be applied to the cholesterol deposit-type atherosclerosis, on the other hand, the local stenting is not effective for the triglyceride deposit-type atherosclerosis since it is a diffuse atherosclerosis. Therefore, it is useful to present information for differentiating whether the subject is the cholesterol deposit-type atherosclerosis or the triglyceride deposit-type atherosclerosis in order to establish a therapeutic approach.

2-3. Presentation Method of Differentiation Information of Dilated Cardiomyopathy A presentation method of differentiation information of the dilated cardiomyopathy according to the present embodiment comprises a step of comparing the value relating to the triglyceride metabolic capacity in the leucocytes of the subject acquired according to the acquisition method described in the above 1. with the predetermined reference range of the triglyceride metabolic capacity, and suggesting that, if the triglyceride metabolic capacity in the leucocytes of the subject is out of the reference range, the subject is not the dilated cardiomyopathy. The suggesting step may be a step of comparing the value relating to the triglyceride metabolic capacity in the leucocytes of the subject with the predetermined reference range of the triglyceride metabolic capacity, and suggesting that, if the triglyceride metabolic capacity in the leucocytes of the subject is within the reference range, the subject is the dilated cardiomyopathy. Alternatively, the suggesting step may be a step of suggesting whether the subject is the dilated cardiomyopathy or not depending on whether the triglyceride metabolic capacity in the leucocytes of the subject is within or out of the reference range.

The triglyceride deposit cardiomyovasculopathy may need to be differentiated from the dilated cardiomyopathy. A therapeutic approach described later is effective for the triglyceride deposit cardiomyovasculopathy. On the other hand, surgical treatment is required for treating the dilated cardiomyopathy. Therefore, it is important to present information for differentiating between the triglyceride deposit cardiomyovasculopathy and the dilated cardiomyopathy in determining a therapeutic strategy for the subject.

### 2-4. Presentation Method of Efficacy Information on Vasodilation Therapy Using Stent

A presentation method of efficacy information on a vasodilation therapy using a stent according to the present embodiment comprises a step of comparing the value relating to the triglyceride metabolic capacity in the leucocytes of the subject acquired according to the acquisition method described in the above 1. with the predetermined reference range of the triglyceride metabolic capacity, and suggesting that, if the triglyceride metabolic capacity in the leucocytes of the subject is out of the reference range, the vasodilation therapy using the stent is not effective in the subject. The suggesting step may be a step of comparing the value relating to the triglyceride metabolic capacity in the leucocytes of the subject with the predetermined reference range of the triglyceride metabolic capacity, and suggesting that, if the triglyceride metabolic capacity in the leucocytes of the subject is within the reference range, the vasodilation therapy using the stent is effective in the subject. Alternatively, the suggesting step may be a step of suggesting whether the vasodilation therapy using the stent is effective or not depending on whether the triglyceride metabolic capacity in the leucocytes of the subject is within or out of the reference range.

In the embodiment described in the above 2-2, it is differentiated whether the subject is cholesterol deposit-type atherosclerosis or triglyceride deposit-type atherosclerosis, on the other hand, in the present embodiment, it is suggested whether the vasodilating therapy using the stent is effective or not without performing the disease differentiation. By performing this method prior to catheterization for stent implantation, subjects for whom the stenting is not effective can be screened.

2-5. Presentation Method of Efficacy Information on Medium Chain Fatty Acid Composition and/or Composition for Regression of Triglyceride Deposit-Type Atherosclerosis

A presentation method of the efficacy information on a medium chain fatty acid composition and/or a composition for regression of the triglyceride deposit-type atherosclerosis according to the present embodiment comprises a step of comparing the value relating to the triglyceride metabolic capacity in the leucocytes of the subject acquired according to the acquisition method described in the above 1. with the predetermined reference range of the triglyceride metabolic capacity, and suggesting that, if the triglyceride metabolic capacity in the leucocytes of the subject is out of the reference range, administration of the medium chain fatty acid composition and/or administration of the composition for regression of the triglyceride deposit-type atherosclerosis to the subject is effective. The suggesting step may be a step of comparing the value relating to the triglyceride metabolic capacity in the leucocytes of the subject with the predetermined reference range of the triglyceride metabolic capacity, and suggesting that, if the triglyceride metabolic capacity in the leucocytes of the subject is within the reference range, the administration of the medium chain fatty acid composition and/or administration of the composition for regression of the triglyceride deposit-type atherosclerosis to the subject is not effective. Alternatively, the suggesting step may be a step of suggesting whether the administration of the medium chain fatty acid composition and/or administration of the composition for regression of the triglyceride deposit-type atherosclerosis is effective or not depending on whether the triglyceride metabolic capacity in the leucocytes of the subject is within or out of the reference range.

The medium chain fatty acid is also referred to as a medium chain triglyceride (MCT). The medium chain fatty acid composition is an oral composition or a parenteral composition comprising a medium chain fatty acid such as pentanoic acid (C5: valeric acid), hexanoic acid (C6: caproic acid), heptanoic acid (C7: enanthic acid), octanoic acid (C8: caprylic acid), nonanoic acid (C9: pelargonic acid), decanoic acid (C10: capric acid), dodecanoic acid (C12: lauric acid) as an active ingredient. The medium chain fatty acid composition is effective for treating or preventing the triglyceride deposit cardiomyovasculopathy or the triglyceride deposit-type atherosclerosis, or preventing aortic aneurysm. Thus, the medium chain fatty acid composition may include a pharmaceutical composition or a food and drink composition.

The composition for regression of the triglyceride deposit-type atherosclerosis is the oral composition or the parenteral composition comprising tricaprin as an active ingredient. The tricaprin as the active ingredient is triacylglycerol constituted by only a medium chain fatty acid having 10 carbon atoms as a constituent fatty acid. That is, the triacylglycerol is a triacylglycerol in which three molecules of capric acid (decanoic acid) are bonded to glycerol by an ester bond. The form of the tricaprin is not limited, and may be in a liquid form, a solid form, or a powder form. The dose of the composition for regression of the triglyceride deposit-type atherosclerosis to humans can be appropriately selected according to the age, sex, weight, severity of disease, or the like of the patient. Usually, the dose of the tricaprin as the active ingredient is selected from the range of 1.0 g to 10.0 g per day, preferably selected from the range of 1.5 g to 9.0 g per day. The number of administrations per day may be one or may be divided into several times.

For an administration period of the composition for regression of the triglyceride deposit-type atherosclerosis, end timing of the administration is not particularly limited and may be appropriately determined by evaluating the regression effect to the triglyceride deposit-type atherosclerosis over time. It is preferred to use the composition to be administered for at least 50 days. The administration period may be two months or more, three months or more, four months or more, five months or more, six months or more, seven months or more, eight months or more, nine months or more, ten months or more, eleven months or more, or one year or more.

The medium chain fatty acid composition and/or the composition for regression of the triglyceride deposit-type atherosclerosis can be administered to a subject who has developed a disease to relieve or cure symptoms caused by the disease. In addition, the medium chain fatty acid composition and/or the composition for regression of the triglyceride deposit-type atherosclerosis can be administered to a subject in the preliminary group of the disease to reduce the risk of developing the disease. The effect of the medium chain fatty acid composition is shown by a reduced incidence of aortic aneurysms in rats fed with a high-fat diet. The effect of the composition for regression of a triglyceride deposit-type atherosclerosis is shown by regression of the triglyceride deposit-type atherosclerosis in a coronary artery in an instance of administration of the composition to a patient with a genetic ATGL (Adipose triglyceride lipase) deficiency and an instance of administration of the composition to a patient with refractory angina pectoris.

### 2-6. Presentation Method of Therapeutic Effect Information

In a presentation method of therapeutic effect information according to the present embodiment, information on therapeutic effect in a subject who has already started being treated with the medium chain fatty acid composition, the composition for regression of a triglyceride deposit-type atherosclerosis, or the like is presented. The presentation method comprises a step of comparing the value relating to the triglyceride metabolic capacity in the leucocytes of the subject acquired according to the acquisition method described in the above 1. with the value relating to the triglyceride metabolic capacity in the leucocytes of the same subject in the past, and suggesting that the therapy applied to the subject is effective when the value relating to the triglyceride metabolic capacity in the leucocytes of the subject is higher than the value relating to the triglyceride metabolic capacity in the leucocytes of the same subject in the past. According to this presentation method, the suggesting step may be a step of comparing the value relating to the triglyceride metabolic capacity in the leucocytes of the subject with the value relating to the triglyceride metabolic capacity in the leucocytes of the subject in the past, and suggesting that the therapy applied to the subject is not effective when the value relating to the triglyceride metabolic capacity in the leucocytes of the subject is equivalent to or lower than the value relating to the triglyceride metabolic capacity in the leucocytes of the subject in the past. Alternatively, the suggesting step may be a step of suggesting whether the treatment applied to the subject is effective or not, depending on whether the value relating to the triglyceride metabolic capacity in the leucocytes of the subject is higher than the value relating to the triglyceride metabolic capacity in the leucocytes of the subject in the past or equivalent to or lower than the value relating to the triglyceride metabolic capacity in the leucocytes of the subject in the past.

### 3. Fluorescence-Labeled Fatty Acid Compound

### 3-1. Structure of Fluorescence-Labeled Fatty Acid Compound

In this section, the fluorescence-labeled fatty acid compound used in the present specification will be described.

The fatty acid compound contains a fatty acid residue, wherein the fatty acid residue has 8 to 26 carbon atoms. The fatty acid residue refers to an acyl group portion of the fatty acid. Here, in the present description, carbon atoms of the fatty acid residue are numbered by an integer, in order from the carbon atom of the carboxy group of the fatty acid residue set to the first position to the terminal methyl group of the fatty acid residue. In the present description, the fatty acid residue may be a saturated fatty acid residue or an unsaturated fatty acid residue. Preferably, it is the saturated fatty acid residue. A part of hydrogen atoms constituting the fatty acid residue may be substituted with an alkyl group having 1 to 3 carbon atoms except for a terminal methyl group of the fatty acid residue. This substitution is referred to as a first substitution. Examples of the alkyl group include a methyl group, an ethyl group, an n-propyl group, an isopropyl group and the like. The alkyl group is preferably the methyl group, the ethyl group, or the n-propyl group, and more preferably the methyl group. The first substitution is conducted at, for example, three sites, preferably two sites, and more preferably one site. The first substitution may be at any hydrogen atoms constituting the fatty acid residue, however, it is preferable that any hydrogen atom bound to carbon atoms at the positions from 2 to 7 is substituted. More preferably, any hydrogen atoms bound to the carbon atom at the positions 2 to 4 is substituted. Particularly preferably, the first substitution is conducted at one site and is a substitution of a hydrogen atom bonded to the carbon atom at the position 3.

The hydrogen atom of the terminal methyl group of the fatty acid residue may be substituted with a substituted or unsubstituted phenyl group. The substitution of the hydrogen atom of the terminal methyl group of the fatty acid residue is referred to as a second substitution. When the phenyl group is substituted, examples of the substituent include an alkyl group having 1 to 3 carbon atoms. The alkyl group for substituting the phenyl group is preferably a methyl group, an ethyl group, an n-propyl group, or an isopropyl group.

The fatty acid compound is preferably a compound represented by following general formula 1:

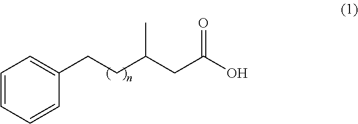

(n is an integer from 4 to 22).

In the compound represented by the general formula 1, n is preferably 8 to 20, more preferably 10 to 18, and particularly preferably 11.

It is preferable that the fatty acid compound is labelled with a fluorescent substance on the terminal methyl group of the fatty acid residue or on the substituent of the methyl group.

The fluorescent substance is not limited as long as it emits fluorescence. However, since a component that emits fluorescence is contained in blood as a part of the blood component, it is preferable that a peak of a fluorescence wavelength of this component and a peak of a fluorescence wavelength of the fluorescent substance with which the fatty acid compound is labelled do not overlap each other. Therefore, the fluorescent substance preferably has a peak of a fluorescence wavelength in a range of 400 nm to 560 nm, or a peak of a fluorescence wavelength in a range of 600 nm to 810 nm, for example. Examples of the fluorescent substance include Alexa Fluor™ 488, FITC, fluorescein, Cy3, Cy2, Alexa Fluor™ 546, Alexa Fluor™ 555, Alexa Fluor™ 633, Alexa Fluor™ 647, Alexa Fluor™ 680, Alexa Fluor™ 700, Alexa Fluor™ 750, Alexa Fluor™ 790, rhodamine, Cy5 and equivalents thereof.

3-2. Production of Fatty Acid Compound to be Labelled with Fluorescent Substance

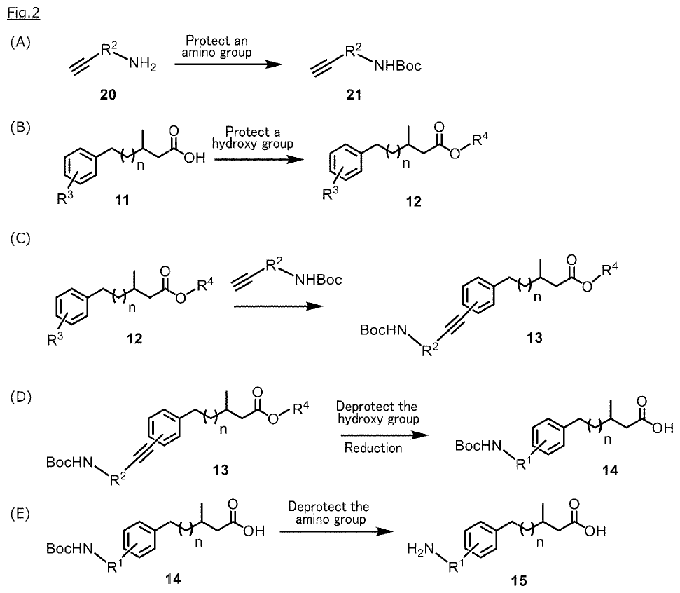

A method for producing the fatty acid compound to be labelled with the fluorescent substance is shown in FIG. 2. The fatty acid compound to be labelled with the fluorescent substance is represented by the following general formula 3:

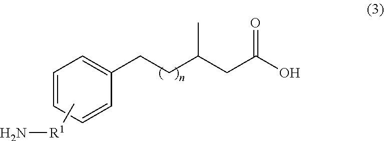

(n is the integer from 4 to 22. R¹ represents an alkylene group having 3 to 6 carbon atoms).

In a synthesis step Ia shown in FIG. 2(A), an amino group of a compound 20 is protected with a protecting group to produce a compound 21. In FIG. 2, the amino acid protecting group is exemplarily represented by Boc. The Boc represents a tert-butoxycarbonyl protecting group. In FIG. 2(A), R² represents an alkylene group having 1 to 4 carbon atoms. This step can be performed by a known method. For example, the compound 20 is dissolved in a solvent such as dichloromethane, mixed with Di-tert-butyl dicarbonate at around 0° C., slowly raised to room temperature, and reacted at about 23° C. to about 28° C. for about 2 hours to allow to produce the compound 21.

In a synthesis step Ib shown in FIG. 2(B), a hydroxy group of a compound 11 is protected with a protecting group to produce a compound 12. In FIG. 2, R³ represents a halogen atom. The halogen atom is preferably an iodine atom. R⁴ represents a protecting group of the hydroxy group. The protecting group of the hydroxy group is preferably a phenyl group. The hydroxy group can be protected by a known method. For example, the compound 11 can be dissolved in a solvent such as triethylamine, benzyl chloride is added thereto, and the mixture is reacted at, for example, about 90° C. for about 2 hours to produce the compound 12.

In a synthesis step II shown in FIG. 2(C), the compound 12 is reacted with the compound 21 to produce a compound 13. For example, the compound 12 is dissolved in tetrahydrofuran, bis(triphenylphosphine)palladium(II) dichloride is added thereto and mixed, the compound 21 dissolved in tetrahydrofuran is further added thereto, and the mixture is stirred at about 23° C. to 28° C. for several minutes. Copper iodide is further added to the stirred mixture, and the mixture is stirred at about 23° C. to 28° C. overnight to produce the compound 13.

In a synthesis step III shown in FIG. 2(D), the compound 13 is reduced to produce a compound 14. This reduction reaction is known. For example, the compound 13 is dissolved in a solvent such as methanol, 10% Pd/C is added thereto, and the mixture is stirred at about 23° C. to 28° C. overnight under a hydrogen atmosphere to produce the compound 14. The compound 14 is a compound represented by general formula 4.

In a synthesis step IV shown in FIG. 2(E), the protecting group of the amino group is removed from the compound 14 to produce a compound 15. This reaction is known. For example, the compound 14 is dissolved in dichloromethane, trifluoroacetic acid is added thereto, and the mixture is stirred at about 23° C. to 28° C. overnight to allow to produce the compound 15.

### 3-3. Fluorescent Substance-Labeling of Fatty Acid Compound

The compound 15 can be labelled with the fluorescent substance by, for example, an amine coupling reaction. For example, an NHS group of a N-hydroxysuccinimide (NHS) ester as the fluorescent substance can be bonded to the amino group of the compound 15 by amine coupling by a known method. The N-hydroxysuccinimide (NHS) ester as the fluorescent substance can be purchased from Thermo Fisher Scientific Inc. or the like.

Preferred aspect of the fluorescence-labeled fatty acid compound is as shown in the following general formula 2:

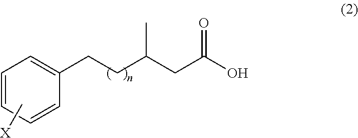

(n is the integer from 4 to 22. X includes the fluorescent substance and a linker moiety).

In the above formula, the linker represents an alkylene group having 3 to 6 carbon atoms, preferably an alkylene group having 3 or 4 carbon atoms, particularly preferably an alkylene group having 3 carbon atoms.

Further preferred aspect of the fluorescence-labeled fatty acid compound is as shown in the following general formula 2′:

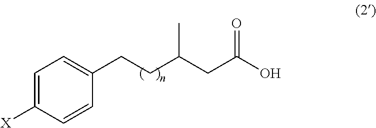

(n is the integer from 4 to 22. X includes the fluorescent substance and the linker moiety).

In the above formula, the linker represents the alkylene group having 3 to 6 carbon atoms, preferably the alkylene group having 3 or 4 carbon atoms, particularly preferably the alkylene group having 3 carbon atoms.

### 4. Test Reagent

The present embodiment relates to a test reagent for evaluating the triglyceride metabolic capacity, which comprises the fatty acid compound labeled with the fluorescent substance. The test reagent may be a state in which the fatty acid compound labeled with the fluorescent substance is dissolved in a solvent such as dimethyl sulfoxide.

### 5. Blood Collection Tube

The present embodiment relates to a blood collection tube used for blood collection for collecting leucocytes of the subject.

The leucocytes collected by the blood collection tube of the present embodiment are used for acquiring the value relating to the triglyceride metabolic capacity described in the above 1 in vitro. The blood collection tube may contain the specific gravity liquid for isolating the leucocytes described in the above 1, or the specific gravity liquid for isolating the leucocytes and the leucocyte separating agent described in the above 1. The blood collection tube is preferably a vacuum blood collection tube, and is preferably attachable to a holder for vacuum blood collection.

Further, the blood collection tube may be provided as a test kit together with the test reagent.

### 6. Presentation System and Presentation Device

An embodiment of the present disclosure relates to a presentation system and a presentation device for executing the presentation method described in the above 2. using a computer.

### 6-1. Configuration of Disease Information Presentation System and Disease Information Presentation Device

### (i) Configuration of Presentation System 1000

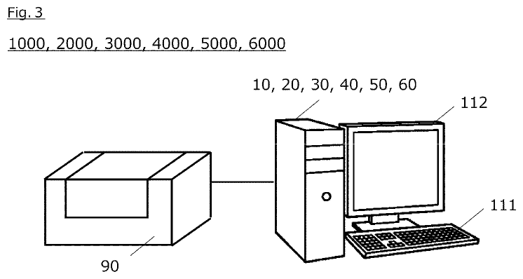

FIG. 3 shows an outline drawing of disease information presentation device system 1000 (hereinafter, simply referred to as a presentation system 1000), and the presentation system 1000 may include, as one aspect, an analysis device 90 in addition to a disease information presentation device 10 (hereinafter, simply referred to as a presentation device 10). The analysis device 90 may be a flow cytometer or a fluorometer.

### (ii) Hardware Configuration of Presentation Device 10

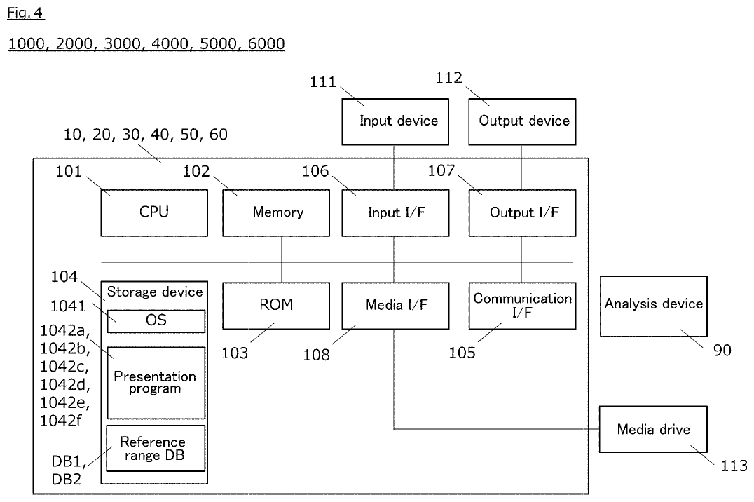

FIG. 4 shows a hardware of the presentation device 10. The presentation device 10 may be connected to an input device 111, an output device 112, and a media drive 113.

In the presentation device 10, a processing unit 101, a memory 102, a read only memory (ROM) 103, a storage device 104, a communication interface (I/F) 105, an input interface (I/F) 106, an output interface (I/F) 107, and a media interface (I/F) 108 are connected to each other via a bus 109 so as to be capable of data communication with each other. The memory 102 and the storage device 104 may be collectively referred to simply as a storage unit. The storage unit stores the measurement value and the reference value in a volatile or nonvolatile manner.

The processing unit 101 is a CPU of the presentation device 10 and is also referred to as a computing device. The processing unit 101 may cooperate with a GPU. The processing unit 101 executes a disease information presentation program 1042a (hereinafter, simply referred to as a presentation program 1042a) described later in cooperation with an operation system (OS) 1041 stored in the storage device 104 or the ROM 103, and processes acquired data, so that the computer functions as the presentation device 10.

The ROM 103 is configured as a mask ROM, a PROM, an EPROM, an EEPROM, or the like, and records the presentation program 1042a executed by the processing unit 101 and data used for the presentation program. The processing unit 101 may be a MPU 101. The ROM 103 stores a boot program executed by the processing unit 101 and a program and settings related to the hardware operation of the presentation device 10 at the time of starting the presentation device 10.

The memory 102 is configured as a random access memory (RAM) such as an SRAM or a DRAM. The memory 102 is used to read the presentation programs 1042a recorded in the ROM 103 and the storage device 104. The memory 102 is used as a work area when the processing unit 101 executes these presentation programs 1042a.

The storage device 104 is configured as a semiconductor memory element such as a hard disk or a flash memory, an optical disk, or the like. The storage device 104 stores various presentation programs 1042a for causing the processing unit 101 to execute, such as an operating system and an application program, and various setting data used for executing the presentation programs 1042a. Specifically, the reference range is stored in a nonvolatile manner as a reference range database (DB) DB1 in which the reference range is recorded.

The communication I/F105 is configured as a serial interface such as USB, IEEE 1394, or RS-232C, a parallel interface such as SCSI, IDE, or IEEE 1284, an analog interface including a D/A converter, an A/D converter, and the like, a network interface controller (NIC), or the like. The communication I/F105 receives data from the analysis device 90 or other external device, and transmits or displays information stored or generated by the presentation device 10 to the analysis device 90 or the outside as necessary, under the control of the processing unit 101. The communication I/F105 may communicate with the analysis device 90 or other external device via a network.

The input I/F106 is configured as, for example, a serial interface such as USB, IEEE 1394, or RS-232C, a parallel interface such as SCSI, IDE, or IEEE 1284, or an analog interface including a D/A converter, an A/D converter, and the like. The input I/F106 receives character input, click input, voice input, and the like from the input device 111. The received input content is stored in the memory 102 or the storage device 104.

The input device 111 is configured as a touch panel, a keyboard, a mouse, a pen tablet, a microphone, or the like, and performs character input or voice input to the presentation device 10. The input device 111 may be connected from the outside of the presentation device 10 or may be integrated with the presentation device 10.

The output I/F107 is configured as, for example, an interface similar to the input I/F106. The output I/F107 outputs the information generated by the processing unit 101 to the output device 112. The output I/F107 outputs the information generated by the processing unit 101 and stored in the storage device 104 to the output device 112.

The output device 112 is configured as, for example, a display, a printer, or the like, and displays measurement results transmitted from the analysis device 90, various operation windows in the presentation device 10, analysis results, and the like.

The media I/F 108 reads, for example, application software or the like stored in the media drive 113. The read application software or the like is stored in the memory 102 or the storage device 104. The media I/F108 writes the information generated by the processing unit 101 into the media drive 113. The media I/F108 writes the information generated by the processing unit 101 and stored in the storage device 104 into the media drive 113.

The media drive 113 is configured as a flexible disk, a CD-ROM, a DVD-ROM, or the like. The media drive 113 is connected to the media I/F108 by a flexible disk drive, a CD-ROM drive, a DVD-ROM drive, or the like. The media drive 113 may store an application program or the like by which the computer executes an operation.

The processing unit 101 may acquire the application software and the various settings necessary for control of the presentation device 10 via a network instead of reading the application software and the various settings from the ROM 103 or the storage device 104. The application program is stored in a storage device of a server computer on a network, and the presentation device 10 can access the server computer to download the presentation program 1042a and store the program in the ROM 103 or the storage device 104.

In addition, an operation system that provides a graphical user interface environment such as Windows® manufactured and sold by Microsoft Corporation is installed in the ROM 103 or the storage device 104, for example. The application program according to a second embodiment is assumed to be operated on the operating system. That is, the presentation device 10 may be a personal computer or the like.

The presentation system 1000 does not need to be installed at one place, and the presentation device 10 and the analysis device 90 may be installed at different places, and these may be connected via a network. The presentation device 10 may be a device with which the input device 111 and the output device 112 are not connected or integrated and which thus does not require an operator.

(iii) Functional Configuration of Presentation Device 10

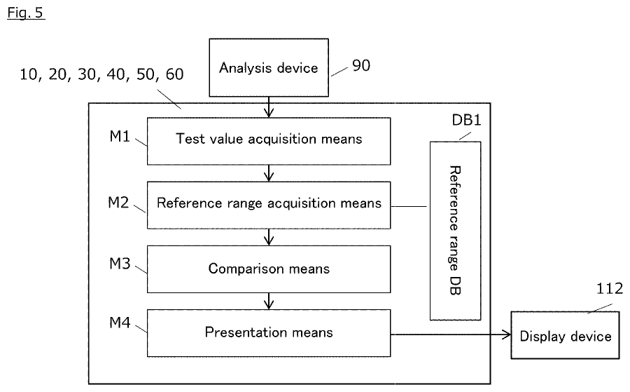

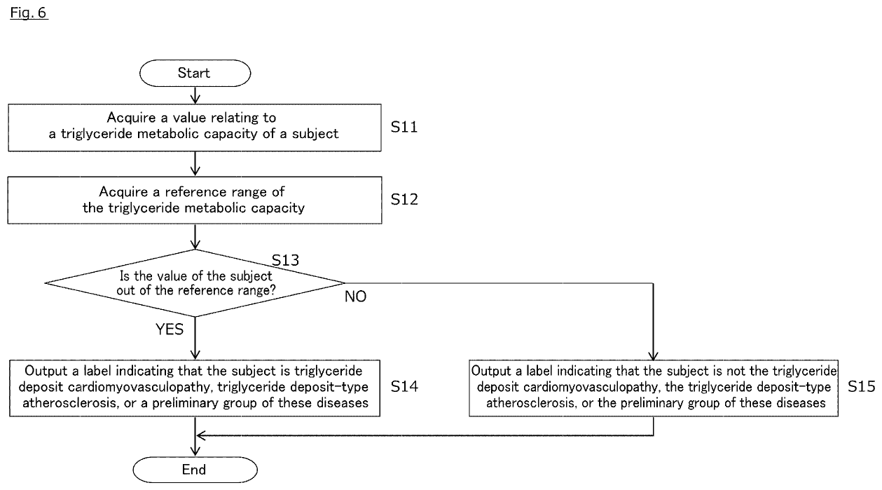

FIG. 5 shows a functional configuration of the presentation device 10. The presentation device 10 comprises a test value acquisition means M1, a reference range acquisition means M2, a comparison means M3, and a presentation means M4. The test value acquisition means M1 corresponds to a step S11 shown in FIG. 6, the reference range acquisition means M2 corresponds to a step S12 shown in FIG. 6, the comparison means M3 corresponds to a step S13 shown in FIG. 6, and the presentation means M4 corresponds to a step S14 shown in FIG. 6.

### (iv) Processing of Disease Information Presentation Program

FIG. 6 shows an example of a flowchart of processing of a disease information presentation program 1042a (hereinafter, simply referred to as a presentation program 1042a).

The processing unit 101 of the presentation device 10 starts processing for presenting disease information due to input of starting processing from the input device 111 by the operator. In the step S11, the processing unit 101 acquires the value relating to the triglyceride metabolic capacity in the leucocytes of the subject from the analysis device 90. Alternatively, the processing unit 101 may acquire the value by receiving an input of the value relating to the triglyceride metabolic capacity in the leucocytes of the subject which is input by the operator from the input device 111.

Next, in the step S12, the processing unit 101 acquires the predetermined reference range of the triglyceride metabolic capacity stored in the reference range database DB1.

Next, in the step S13, the processing unit 101 compares the value relating to the triglyceride metabolic capacity acquired in the step S11 with the predetermined reference range acquired in the step S12.

In the step S13, when the acquired value relating to the triglyceride metabolic capacity is out of the reference range (in the case of YES), the process proceeds to step S14, where the processing unit 101 determines that the subject is the triglyceride deposit cardiomyovasculopathy, the triglyceride deposit-type atherosclerosis, or the preliminary group of these diseases, and outputs a label indicating that the subject is the triglyceride deposit cardiomyovasculopathy, the triglyceride deposit-type atherosclerosis, or the preliminary group of these diseases as the disease information to the output device 112. In the step S13, when the acquired value relating to the triglyceride metabolic capacity is within the reference range (in the case of NO), the process proceeds to a step S15, where the processing unit 101 determines that the subject is not the triglyceride deposit cardiomyovasculopathy, the triglyceride deposit-type atherosclerosis, or the preliminary group of these diseases, and outputs a label indicating that the subject is not the triglyceride deposit cardiomyovasculopathy, the triglyceride deposit-type atherosclerosis, or the preliminary group of these diseases as the disease information to the output device 112. The label output in the step S14 includes information indicating that “the subject is the triglyceride deposit cardiomyovasculopathy, the triglyceride deposit-type atherosclerosis, or the preliminary group of these diseases” or “it is suggested that the subject is the triglyceride deposit cardiomyovasculopathy, the triglyceride deposit-type atherosclerosis, or the preliminary group of these diseases”. The label output in the step S15 includes information indicating that “the subject is not the triglyceride deposit cardiomyovasculopathy, the triglyceride deposit-type atherosclerosis, or the preliminary group of these diseases” or “it is not suggested that the subject is the triglyceride deposit cardiomyovasculopathy, the triglyceride deposit-type atherosclerosis, or the preliminary group of these diseases”. The information may be a mark such as ×, ∘, an exclamation mark, or the like.

For detailed description of the value relating to the triglyceride metabolic capacity, the reference range, the comparison method, the determination method, and the like, the description in the above 2. is incorporated herein by reference.

6-2. Configurations of Presentation System for Differentiation Information on Cholesterol Deposit-Type Atherosclerosis or Preliminary Group Thereof and Presentation Device for Differentiation Information on Cholesterol Deposit-Type Atherosclerosis or Preliminary Group Thereof

### (i) Configuration of Presentation System 2000

The appearance of a presentation system 2000 for differentiation information on the cholesterol deposit-type atherosclerosis or the preliminary group thereof (hereinafter simply referred to as a presentation system 2000) is similar to that of the presentation system 1000 shown in FIG. 3, and thus, the description in the above 6-1. (i) is incorporated herein by reference. Provided that, the presentation device 10 in FIG. 3 is to be read as a presentation device 20 for differentiation information on the cholesterol deposit-type atherosclerosis or the preliminary group thereof (hereinafter simply referred to as a presentation device 20).

### (ii) Hardware Configuration of Presentation Device 20

The hardware configuration of the presentation device 20 is similar to the presentation device 10 shown in FIG. 4 except that a presentation program 1042b for differentiation information of the dilated cardiomyopathy is stored in the storage device 104, and thus, the description in the above 6-1. (ii) is incorporated herein by reference.

(iii) Functional Configuration of Presentation Device 20

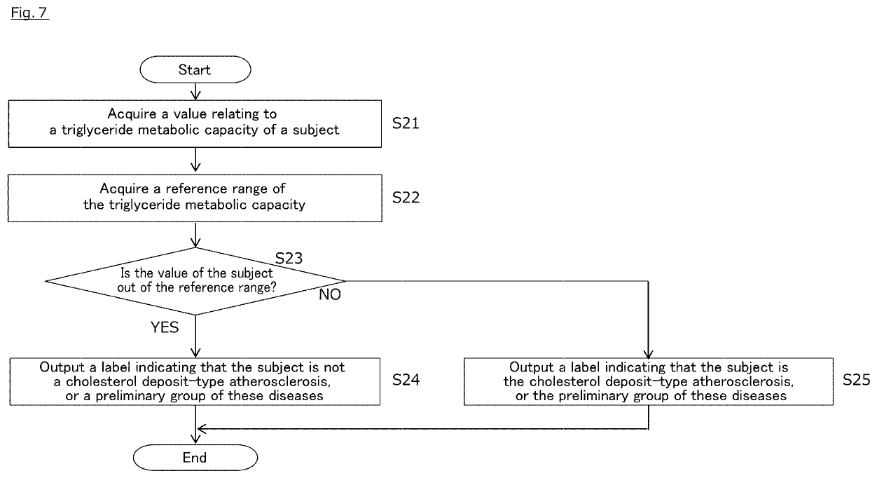

The functional configuration of the presentation device 20 is basically the same as that of presentation device 10 shown in FIG. 5. Provided that, the test value acquisition means M1 corresponds to a step S21 shown in FIG. 7, the reference range acquisition means M2 corresponds to a step S22 shown in FIG. 7, the comparison means M3 corresponds to a step S23 shown in FIG. 7, the presentation means M4 corresponds to a step S24 shown in FIG. 7.

### (iv) Processing of Presentation Program for Differentiation Information on Cholesterol Deposit-Type Atherosclerosis or Preliminary Group Thereof

FIG. 7 shows an example of a flowchart of processing of a presentation program 1042b for differentiation information on the cholesterol deposit-type atherosclerosis or the preliminary group thereof (hereinafter, simply referred to as the presentation program 1042b).

The processing unit 101 of the presentation device 20 starts processing differentiation information on the cholesterol deposit-type atherosclerosis or the preliminary group thereof due to input of starting processing from the input device 111 by the operator. In the step S21, the processing unit 101 acquires the value relating to the triglyceride metabolic capacity in the leucocytes of the subject from the analysis device 90. Alternatively, the processing unit 101 may acquire the value by receiving an input of the value relating to the triglyceride metabolic capacity in the leucocytes of the subject which is input by the operator from the input device 111.

Next, in the step S22, the processing unit 101 acquires the predetermined reference range of the triglyceride metabolic capacity stored in the reference range database DB1.

Next, in step the S23, the processing unit 101 compares the value relating to the triglyceride metabolic capacity acquired in the step S21 with the predetermined reference range acquired in the step S22.

In the step S23, when the acquired value relating to the triglyceride metabolic capacity is out of the reference range (in the case of YES), the process proceeds to the step S24, where the processing unit 101 determines that the subject is not the cholesterol deposit-type atherosclerosis or the preliminary group thereof, and outputs a label indicating that the subject is not the cholesterol deposit-type atherosclerosis or the preliminary group thereof as the disease information to the output device 112. In the step S23, when the acquired value relating to the triglyceride metabolic capacity is within the reference range (in the case of NO), the process proceeds to a step S25, where the processing unit 101 determines that the subject is the cholesterol deposit-type atherosclerosis or the preliminary group thereof, and outputs a label indicating that the subject is the cholesterol deposit-type atherosclerosis or the preliminary group thereof as the differentiation information to the output device 112. The label output in the step S24 includes information indicating that “the subject is not the cholesterol deposit-type atherosclerosis or the preliminary group thereof” or “it is not suggested that the subject is the cholesterol deposit-type atherosclerosis or the preliminary group thereof”. The label output in the step S25 includes information indicating that “the subject is the cholesterol deposit-type atherosclerosis or the preliminary group thereof” or “it is suggested that the subject is the cholesterol deposit-type atherosclerosis or the preliminary group thereof”. The information may be a mark such as ×, ∘, an exclamation mark, or the like.

For detailed description of the value relating to the triglyceride metabolic capacity, the reference range, the comparison method, the determination method, and the like, the description in the above 2. is incorporated herein by reference.

### 6-3. Configurations of Presentation System for Differentiation Information on Dilated Cardiomyopathy and Presentation Device for Differentiation Information on Dilated Cardiomyopathy

### (i) Configuration of Presentation System 3000

The appearance of a presentation system 3000 for differentiation information on the dilated cardiomyopathy (hereinafter simply referred to as a presentation system 3000) is similar to that of the presentation system 1000 shown in FIG. 3, and thus, the description in the above 6-1. (i) is incorporated herein by reference. Provided that, the presentation device 10 in FIG. 3 is to be read as a presentation device 30 for differentiation information on the dilated cardiomyopathy (hereinafter simply referred to as a presentation device 30).

### (ii) Hardware Configuration of Presentation Device 30

The hardware configuration of the presentation device 30 is similar to the presentation device 10 shown in FIG. 4 except that a presentation program 1042c for differentiation information of the dilated cardiomyopathy is stored in the storage device 104, and thus, the description in the above 6-1. (ii) is incorporated herein by reference.

(iii) Functional Configuration of Presentation Device 30

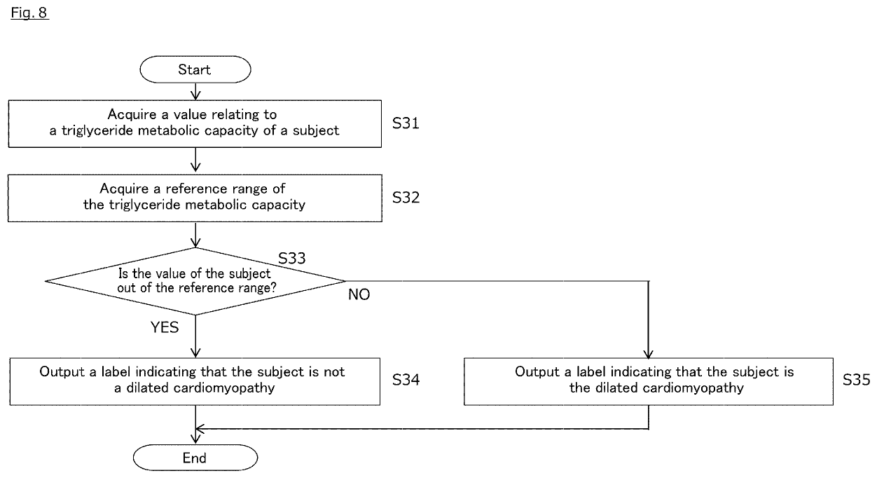

The functional configuration of the presentation device 30 is basically the same as that of the presentation device 10 shown in FIG. 5. Provided that, the test value acquisition means M1 corresponds to a step S31 shown in FIG. 8, the reference range acquisition means M2 corresponds to a step S32 shown in FIG. 8, the comparison means M3 corresponds to a step S33 shown in FIG. 8, the presentation means M4 corresponds to a step S34 shown in FIG. 8.

### (iv) Processing of Presentation Program for Differentiation Information on Dilated Cardiomyopathy

FIG. 8 shows an example of a flowchart of processing of a presentation program 1042c for differentiation information on the dilated cardiomyopathy (hereinafter, simply referred to as a presentation program 1042c).

The processing unit 101 of the presentation device 30 starts processing for presenting differentiation information on the dilated cardiomyopathy due to input of starting processing from the input device 111 by the operator. In the step S31, the processing unit 101 acquires the value relating to the triglyceride metabolic capacity in the leucocytes of the subject from the analysis device 90. Alternatively, the processing unit 101 may acquire the value by receiving an input of the value relating to the triglyceride metabolic capacity in the leucocytes of the subject which is input by the operator from the input device 111.

Next, in the step S32, the processing unit 101 acquires the predetermined reference range of the triglyceride metabolic capacity stored in the reference range database DB1.

Next, in step the S33, the processing unit 101 compares the value relating to the triglyceride metabolic capacity acquired in the step S31 with the predetermined reference range acquired in the step S32.

In the step S33, when the acquired value relating to the triglyceride metabolic capacity is out of the reference range (in the case of YES), the process proceeds to the step S34, where the processing unit 101 determines that the subject is not the dilated cardiomyopathy, and outputs a label indicating that the subject is not the dilated cardiomyopathy as the differentiation information to the output device 112. In the step S33, when the acquired value relating to the triglyceride metabolic capacity is within the reference range (in the case of NO), the process proceeds to a step S35, where the processing unit 101 determines that the subject is the dilated cardiomyopathy, and outputs a label indicating that the subject is the dilated cardiomyopathy as the differentiation information to the output device 112. The label output in the step S34 includes information indicating that “the subject is not the dilated cardiomyopathy” or “it is not suggested that the subject is the dilated cardiomyopathy”. The label output in the step S35 includes information indicating that “the subject is the dilated cardiomyopathy” or “it is suggested that the subject is the dilated cardiomyopathy”. The information may be a mark such as ×, ∘, an exclamation mark, or the like.

For detailed description of the value relating to the triglyceride metabolic capacity, the reference range, the comparison method, the determination method, and the like, the description in the above 2. is incorporated herein by reference.

### 6-4. Configurations of Presentation System for Efficacy Information on Vasodilation Therapy Using Stent and Presentation Device for Efficacy Information on Vasodilation Therapy Using Stent

### (i) Configuration of Presentation System 4000

The appearance of a presentation system 4000 for efficacy information on a vasodilation therapy using a stent (hereinafter simply referred to as a presentation system 4000) is similar to that of the presentation system 1000 shown in FIG. 3, and thus, the description in the above 6-1. (i) is incorporated herein by reference. Provided that, the presentation device 10 in FIG. 3 is to be read as a presentation device 40 for the efficacy information on the vasodilation therapy using the stent (hereinafter simply referred to as a presentation device 40).

### (ii) Hardware Configuration of Presentation Device 40

The hardware configuration of the presentation device 40 is similar to the presentation device 10 shown in FIG. 4 except that a presentation program 1042d for efficacy information on the vasodilation therapy using the stent is stored in the storage device 104, and thus, the description in the above 6-1. (ii) is incorporated herein by reference.

(iii) Functional Configuration of Presentation Device 40

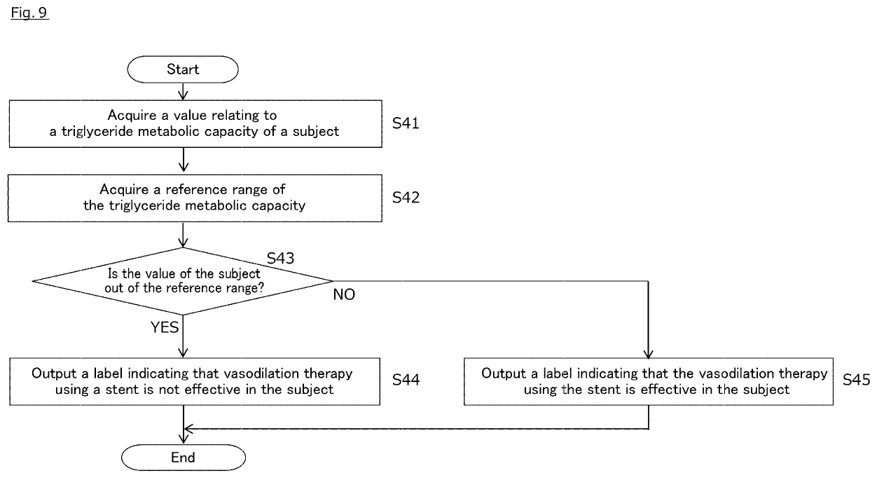

The functional configuration of the presentation device 40 is basically the same as that of the presentation device 10 shown in FIG. 5. Provided that, the test value acquisition means M1 corresponds to a step S41 shown in FIG. 9, the reference range acquisition means M2 corresponds to a step S42 shown in FIG. 9, the comparison means M3 corresponds to a step S43 shown in FIG. 9, the presentation means M4 corresponds to a step S44 shown in FIG. 9.

### (iv) Processing of Presentation Program for Efficacy Information on Vasodilation Therapy Using Stent

FIG. 9 shows an example of a flowchart of processing of a presentation program 1042d for efficacy information on the vasodilation therapy using the stent (hereinafter, simply referred to as a presentation program 1042d).

The processing unit 101 of the presentation device 40 starts processing for presenting efficacy information on the vasodilation therapy using the stent due to input of starting processing from the input device 111 by the operator. In the step S41, the processing unit 101 acquires the value relating to the triglyceride metabolic capacity in the leucocytes of the subject from the analysis device 90. Alternatively, the processing unit 101 may acquire the value by receiving an input of the value relating to the triglyceride metabolic capacity in the leucocytes of the subject which is input from the input device 111 by the operator.

Next, in the step S42, the processing unit 101 acquires the predetermined reference range of the triglyceride metabolic capacity stored in the reference range database DB1.

Next, in step the S43, the processing unit 101 compares the value relating to the triglyceride metabolic capacity acquired in the step S41 with the predetermined reference range acquired in the step S42.

In the step S43, when the acquired value relating to the triglyceride metabolic capacity is out of the reference range (in the case of YES), the process proceeds to the step S44, where the processing unit 101 determines that the vasodilation therapy using the stent is not effective in the subject, and outputs a label indicating that the vasodilation therapy using the stent is not effective in the subject as the efficacy information to the output device 112. In the step S43, when the acquired value relating to the triglyceride metabolic capacity is within the reference range (in the case of NO), the process proceeds to a step S45, where the processing unit 101 determines that the vasodilation therapy using the stent is effective in the subject, and outputs a label indicating that the vasodilation therapy using the stent is effective in the subject as the efficacy information to the output device 112. The label output in the step S44 includes information indicating that “the stenting is not effective” or “it is not suggested that the stenting is effective”. The label output in the step S45 includes information indicating that “the stenting is effective” or “it is suggested that the stenting is effective”. The information may be a mark such as ×, ∘, an exclamation mark, or the like.

For detailed description of the value relating to the triglyceride metabolic capacity, the reference range, the comparison method, the determination method, and the like, the description in the above 2. is incorporated herein by reference.

6-5. Configurations of Presentation System for Efficacy Information on Medium Chain Fatty Acid Composition and/or Composition for Regression of Triglyceride Deposit-Type Atherosclerosis and Presentation Device for Differentiation Information on Medium Chain Fatty Acid Composition and/or Composition for Regression of Triglyceride Deposit-Type Atherosclerosis

### (i) Configuration of Presentation System 5000

The appearance of a presentation system 5000 for efficacy information on a medium chain fatty acid composition and/or a composition for regression of a triglyceride deposit-type atherosclerosis (hereinafter simply referred to as a presentation system 5000) is similar to that of the presentation system 1000 shown in FIG. 3, and thus, the description in the above 6-1. (i) is incorporated herein by reference. Provided that, the presentation device 10 in FIG. 3 is to be read as a presentation device 50 for the efficacy information on the medium chain fatty acid composition and/or the composition for regression of the triglyceride deposit-type atherosclerosis (hereinafter simply referred to as a presentation device 50).

### (ii) Hardware Configuration of Presentation Device 50

The hardware configuration of the presentation device 50 is similar to the presentation device 10 shown in FIG. 4 except that a presentation program 1042e for efficacy information on the vasodilation therapy using the stent is stored in the storage device 104, and thus, the description in the above 6-1. (ii) is incorporated herein by reference.

(iii) Functional Configuration of Presentation Device 50

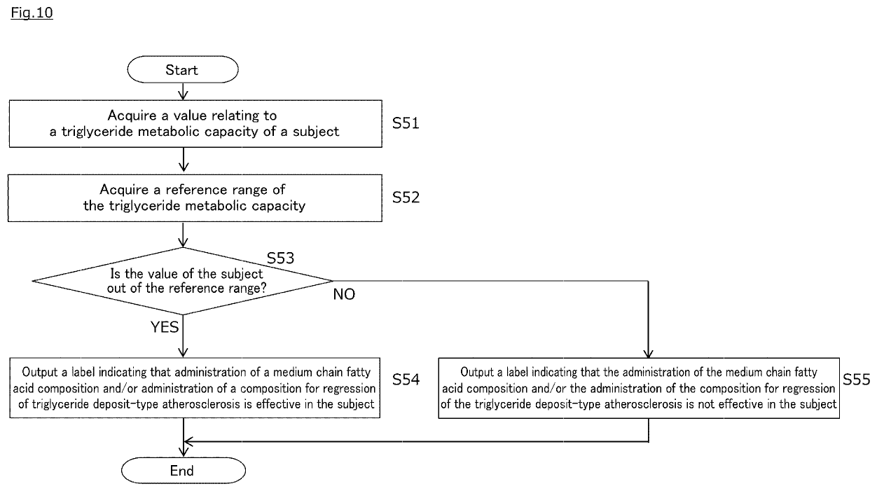

The functional configuration of the presentation device 50 is basically the same as that of the presentation device 10 shown in FIG. 5. Provided that, the test value acquisition means M1 corresponds to a step S51 shown in FIG. 10, the reference range acquisition means M2 corresponds to a step S52 shown in FIG. 10, the comparison means M3 corresponds to a step S53 shown in FIG. 10, the presentation means M4 corresponds to a step S54 shown in FIG. 10.

(iv) Processing of Presentation Program for Efficacy Information on Medium Chain Fatty Acid Composition and/or Composition for Regression of Triglyceride Deposit-Type Atherosclerosis

FIG. 10 shows an example of a flowchart of processing of a presentation program 1042e for efficacy information on the medium chain fatty acid composition and/or the composition for regression of the triglyceride deposit-type atherosclerosis (hereinafter, simply referred to as a presentation program 1042e).

The processing unit 101 of the presentation device 50 starts processing for presenting the efficacy information on the medium chain fatty acid composition and/or the composition for regression of the triglyceride deposit-type atherosclerosis due to input of starting processing from the input device 111 by the operator. In the step S51, the processing unit 101 acquires the value relating to the triglyceride metabolic capacity in the leucocytes of the subject from the analysis device 90. Alternatively, the processing unit 101 may acquire the value by receiving an input of the value relating to the triglyceride metabolic capacity in the leucocytes of the subject which is input from the input device 111 by the operator.

Next, in the step S52, the processing unit 101 acquires the predetermined reference range of the triglyceride metabolic capacity stored in the reference range database DB1.

Next, in step the S53, the processing unit 101 compares the value relating to the triglyceride metabolic capacity acquired in the step S51 with the predetermined reference range acquired in the step S52.

In the step S53, when the acquired value relating to the triglyceride metabolic capacity is out of the reference range (in the case of YES), the process proceeds to the step S54, where the processing unit 101 determines that the administration of the medium chain fatty acid composition and/or the administration of the composition for regression of the triglyceride deposit-type atherosclerosis is effective in the subject, and outputs a label indicating that the administration of the medium chain fatty acid composition and/or the administration of the composition for regression of the triglyceride deposit-type atherosclerosis is effective in the subject as the efficacy information to the output device 112. In the step S53, when the acquired value relating to the triglyceride metabolic capacity is within the reference range (in the case of NO), the process proceeds to a step S55, where the processing unit 101 determines that the administration of the medium chain fatty acid composition and/or the administration of the composition for regression of the triglyceride deposit-type atherosclerosis is not effective in the subject, and outputs a label indicating that the administration of the medium chain fatty acid composition and/or the administration of the composition for regression of the triglyceride deposit-type atherosclerosis is not effective in the subject as the efficacy information to the output device 112. The label output in the step S54 includes information indicating that “the administration of the medium chain fatty acid composition and/or the administration of the composition for regression of the triglyceride deposit-type atherosclerosis is effective” or “it is suggested that the administration of the medium chain fatty acid composition and/or the administration of the composition for regression of the triglyceride deposit-type atherosclerosis is effective”. The label output in the step S55 includes information indicating that “the administration of the medium chain fatty acid composition and/or the administration of the composition for regression of the triglyceride deposit-type atherosclerosis is not effective” or “it is not suggested that the administration of the medium chain fatty acid composition and/or the administration of the composition for regression of the triglyceride deposit-type atherosclerosis is effective”. The information may be a mark such as ×, ∘, an exclamation mark, or the like.

For detailed description of the value relating to the triglyceride metabolic capacity, the reference range, the comparison method, the determination method, and the like, the description in the above 2. is incorporated herein by reference.

### 6-5. Configurations of Presentation System for Therapeutic Effect Information and Presentation Device for Therapeutic Effect Information

### (i) Configuration of Presentation System 6000

The appearance of a presentation system 6000 for therapeutic effect information (hereinafter simply referred to as a presentation system 6000) is similar to that of the presentation system 1000 shown in FIG. 3, and thus, the description in the above 6-1. (i) is incorporated herein by reference. Provided that, the presentation device 10 in FIG. 3 is to be read as a presentation device 60 for the therapeutic effect information (hereinafter simply referred to as a presentation device 60).

### (ii) Hardware Configuration of Presentation Device 60

The hardware configuration of the presentation device 60 is similar to the presentation device 10 shown in FIG. 4 except that a presentation program 1042f for efficacy information on the therapeutic effect information on the stenting and a reference range database (DB) DB2 in which the value relating to the triglyceride metabolic capacity of the subject in the past is recorded are stored in the storage device 104, and thus, the description in the above 6-1. (ii) is incorporated herein by reference.

(iii) Functional Configuration of Presentation Device 60

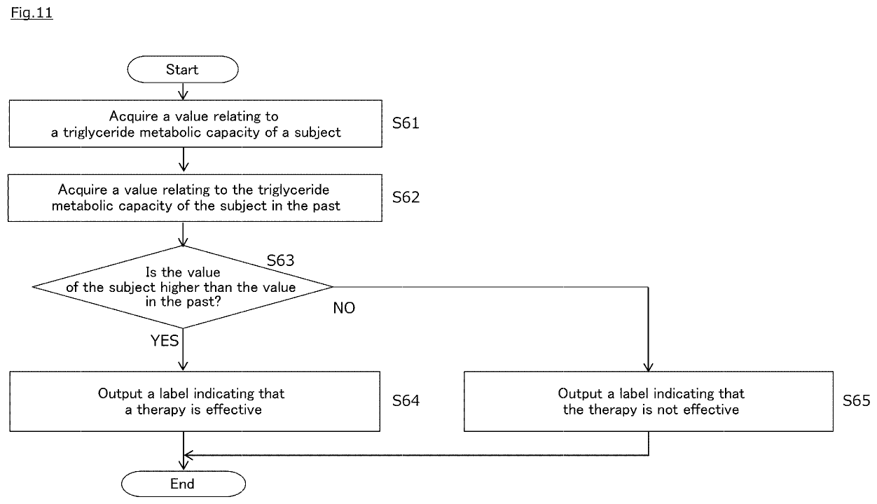

The functional configuration of the presentation device 60 is basically the same as that of the presentation device 10 shown in FIG. 5. Provided that, the test value acquisition means M1 corresponds to a step S61 shown in FIG. 11, the reference range acquisition means M2 corresponds to a step S62 shown in FIG. 11, the comparison means M3 corresponds to a step S63 shown in FIG. 11, the presentation means M4 corresponds to a step S64 shown in FIG. 10.

### (iv) Processing of Presentation Program for Therapeutic Effect Information

FIG. 11 shows an example of a flowchart of processing of a presentation program 1042f for therapeutic effect information (hereinafter, simply referred to as a presentation program 1042f).

The processing unit 101 of the presentation device 60 starts processing for presenting the therapeutic effect information due to input of starting processing from the input device 111 by the operator. In the step S61, the processing unit 101 acquires the value relating to the triglyceride metabolic capacity in the leucocytes of the subject from the analysis device 90. Alternatively, the processing unit 101 may acquire the value by receiving an input of the value relating to the triglyceride metabolic capacity in the leucocytes of the subject which is input from the input device 111 by the operator.

Next, in the step S62, the processing unit 101 acquires the value relating to the triglyceride metabolic capacity of the same subject in the past stored in the reference range database DB2.

Next, in step the S63, the processing unit 101 compares the value relating to the triglyceride metabolic capacity acquired in the step S61 with the value relating to the triglyceride metabolic capacity of the subject in the past acquired in the step S62.

In the step S63, when the acquired value relating to the triglyceride metabolic capacity is higher than the value relating to the triglyceride metabolic capacity of the subject in the past (in the case of YES), the process proceeds to the step S64, where the processing unit 101 determines that a therapy applied to the subject is effective, and outputs a label indicating that the therapy is effective as the therapeutic effect information to the output device 112. In the step S63, when the acquired value relating to the triglyceride metabolic capacity is lower than the value relating to the triglyceride metabolic capacity of the subject in the past (in the case of NO), the process proceeds to a step S65, where the processing unit 101 determines that the therapy applied to the subject is not effective, and outputs a label indicating that the therapy applied to the subject is not effective as the therapeutic effect information to the output device 112. The label output in the step S64 includes information indicating that “the therapy is effective” or “it is suggested that the therapy is effective”. The label output in the step S65 includes information indicating that “the therapy is not effective” or “it is not suggested that the therapy is effective”. The information may be a mark such as ×, ∘, an exclamation mark, or the like.

For detailed description of the value relating to the triglyceride metabolic capacity, the reference range, the comparison method, the determination method, and the like, the description in the above 2. is incorporated herein by reference.

### 6-7. Variants

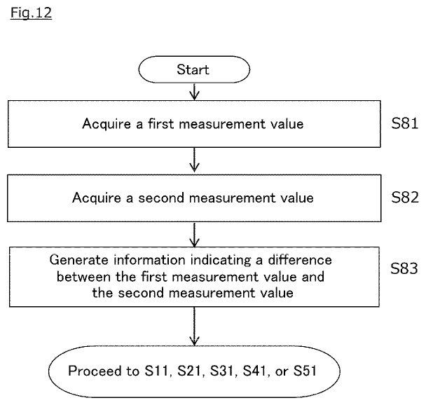

The presentation programs 1042a, 1042b, 1042c, 1042d, 1042e, and 1042f may comprise a process of generating information indicating a difference between the first measurement value and the second measurement value shown in FIG. 12 before each of steps S11, S21, S31, S41, S51, and S61.

In Step S81, the processing unit 101 of each presentation device acquires the first measurement value from the analysis device 90 according to a measurement value acquisition command input which is input by the operator from the input device 111. In addition, in Step S82, the processing unit 101 of each presentation device acquires the second measurement value from the analysis device 90 according to the measurement value acquisition command input which is input by the operator from the input device 111. Further, the processing part 101 of each presentation apparatus generates the information indicating the difference between the first measurement value and the second measurement value, following the command input from the input device 111 by the operator, or by using the second measurement value as a trigger. The information indicating the difference is not limited as long as the information indicates the difference between the first measurement value and the second measurement value. For example, a subtraction value, a division value, a relative value, or the like may be used as the information. Further, the information may be presented by displaying histograms or the like indicating the first measurement value and the second measurement value side by side.

### 7. Storage Device Storing Presentation Program

The present embodiment includes the presentation programs 1042a, 1042b, 1042c, 1042d, 1042e, and 1042f described above.

In addition, an embodiment of the present embodiment relates to a program product such as a media drive that stores the presentation programs 1042a, 1042b, 1042c, 1042d, and 1042e. That is, the presentation programs 1042a, 1042b, 1042c, 1042d, and 1042e may be stored in a semiconductor memory element such as a hard disk or a flash memory, or a media drive such as an optical disk. The media drive may be a computer such as a server device. The recording format of the program on the media drive is not limited as long as the presentation devices 10, 20, 30, 40, and 50 can read the program. It is preferable that the program is recorded on the media drive in a nonvolatile manner.

### EXAMPLES

Hereinafter, the present invention will be described in more detail with reference to examples, but the interpretation of the present invention shall not be limited to the examples.

### 1. Experimental Example 1: Preparation of Fluorescence-Labeled Fatty Acid

In order to verify that a fluorescence-labeled fatty acid was taken into cells as a fatty acid analog and excreted, 15-phenyl-3-methylpentadenoic acid labeled with Alexa Fluor™ 680 (hereinafter, referred to as a fluorescence-labeled BMPP) was prepared.

### 1-1. Preparation of BMPP

15-(4-(3-aminopropyl)phenyl)-3-methylpentadecanoic acid was synthesized by the following method. In the following synthesis system, EtOAc means ethyl acetate, Boc means a tert-butoxycarbonyl protecting group, and MeOH means methanol.

### (i) Step 1a: Synthesis of tert-butyl prop-2-yn-1-ylcarbamate

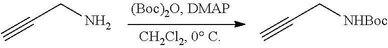

Boc anhydride (4 g, 18.2 mmol) dissolved in CH₂Cl₂ was added to a solution of propargylamine (1 g, 18.2 mmol) in CH₂Cl₂ (25 mL) at 0° C. with stirring. The temperature was slowly raised to room temperature and stirred for another 2 hours. After the reaction was completed, CH₂Cl₂ was evaporated in vacuo and the residue was extracted with EtOAc. The organic layer was dried over MgSO₄ and concentrated in vacuo. The resulting residue was purified by silica gel column chromatography using hexane/EtOAc (8:2) to afford a desired Boc-protected propargylamine (2.4 g, 95% yield) as a light yellow solid.

¹H NMR (CDCl₃, 500 MHz): δ=4.71 (1H, s), 3.92 (2H, s), 2.22 (1H, t, J=2.5 Hz), 1.45 (9H, s)

### (ii) Step 1b: Synthesis of benzyl 15-(4-iodophenyl)-3-methylpentadecanoate (2)

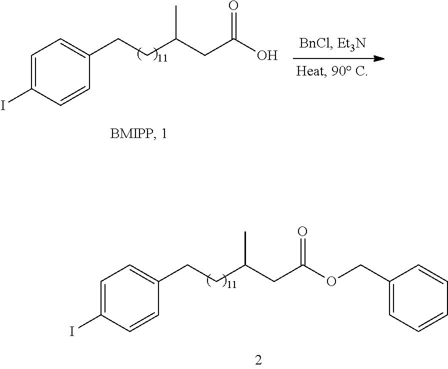

Benzyl chloride (275 mg, 2.2 mmol) was added to a triethylamine solution (about 0.3 mL, 1:1 v/v) of 15-(4-iodophenyl)-3-methylpentadenocanoic acid: BMIPP (200 mg, 0.44 mmol) with stirring. The reaction mixture was stirred at 90° C. for 2 hours. After the reaction was completed, the residue was extracted with EtOAc. The organic layer was dried over MgSO₄ and concentrated in vacuo. The resulting residue was purified by silica gel column chromatography using hexane/EtOAc (9:1) to afford a desired compound 2 (215 mg, 90% yield) as a colorless oil.

¹H NMR (CDCl₃, 500 MHz): δ=7.60 (2H, d, J=8.3 Hz), 7.36 (5H, m), 6.94 (2H, d, J=8.1 Hz), 5.12 (2H, s), 2.54 (2H, t, J=7.8 Hz), 2.36 (1H, dd, J=14.9, 6.1 Hz), 2.17 (1H, dd, J=14.6, 8.0 Hz), 1.56-1.59 (2H, m), 1.24-1.29 (20H, m), 0.93 (3H, d, J=6.6 Hz).

### (iii) Step 2: Synthesis of benzyl 15-(4-(3-((tert-butoxycarbonyl)amino)prop-1-yn-1-yl)phenyl)-3-methylpentadecanoate (3)

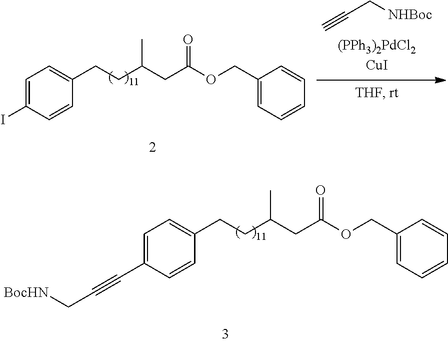

Triethylamine (110 mg, 1.1 mmol) and bis(triphenylphosphine)palladium(II) dichloride (51 mg, 0.073 mmol) was added to a solution of the compound 2 (200 mg, 0.37 mmol) in THF with stirring. The reaction mixture of the compound 2 was stirred at room temperature for several minutes. Boc-protected propargylamine (170 mg, 1.1 mmol) dissolved in THF was slowly added to the reaction mixture of the compound 2 and stirred for 5 minutes. Copper iodide (7 mg, 0.037 mmol) was added and the reaction mixture was stirred at room temperature overnight. After the reaction was completed, the THF was removed in vacuo. The resulting residue was purified by silica gel column chromatography using hexane/EtOAc (8:2) to afford a desired compound 3 (115 mg, 55% yield) as a yellow oil.

¹H NMR (CDCl₃, 500 MHz): δ=7.31-7.38 (7H, m), 7.12 (2H, d, J=8.3 Hz), 5.12 (2H, s), 4.75 (1H, s) 4.15 (2H, s), 2.58 (2H, t, J=7.8 Hz), 2.36 (1H, dd, J=14.6, 6.1 Hz), 2.17 (1H, dd, J=14.6, 8.0 Hz), 1.57-1.62 (2H, m), 1.47 (9H, s), 1.24-1.30 (20H, m), 0.93 (3H, d, J=6.8).

### (iv) Step 3: Synthesis of 15-(4-(3-((tert-butoxycarbonyl)amino)propyl)phenyl)-3-methylpentadecanoic acid (4)

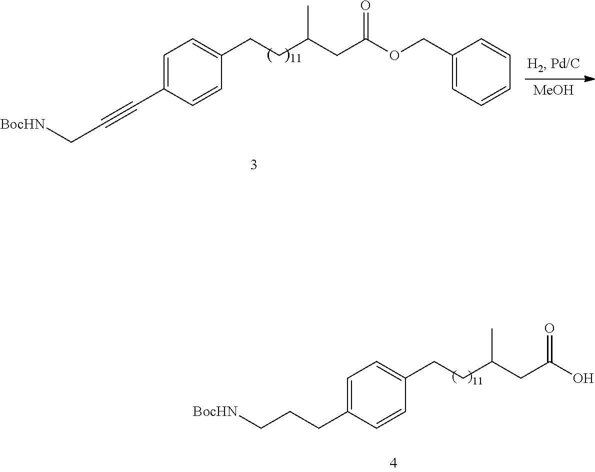

10% Pd/C (120 mg, 0.12 mmol) was added to a solution of the compound 3 (100 mg, 0.174 mmol) in MeOH (10 ml) with stirring. The reaction mixture was stirred at room temperature overnight under H2 atmosphere. The solid was filtered off and the filtrate was concentrated under vacuum to afford a residue, and then the residue was purified by silica gel column chromatography using CHCl₃/MeOH (9.5:0.5) to afford a compound 4 (75 mg, 92% yield) as a colorless oil.

¹H NMR (CDCl₃, 500 MHz): δ=7.09 (4H, m), 4.53 (1H, s), 3.15 (1H, s), 2.61 (2H, t, J=7.7 Hz), 2.57 (2H, t, J=7.3), 2.33-2.37 (1H, dd, J=15.9, 6.1 Hz), 2.16 (1H, dd, J=14.9, 8.0 Hz), 1.93-1.98 (1H, m), 1.77-1.82 (2H, m), 1.56-1.60 (2H, m), 1.45 (9H, s), 1.24-1.30 (20H, m), 0.97 (3H, d, J=6.6 Hz).

### (v) Step 4: Synthesis of 15-(4-(3-aminopropyl)phenyl)-3-methylpentadecanoic acid (5)

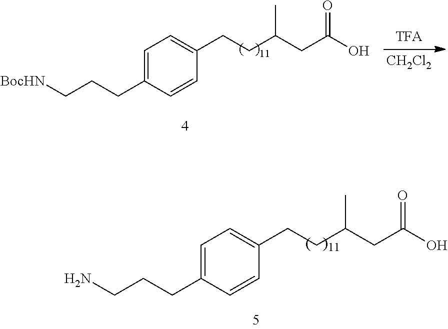

TFA (160 mg, 1.43 mmol) was added to a solution of the compound 4 (70 mg, 0.143 mmol) in CH₂Cl₂(8 mL) with stirring. The reaction mixture was stirred at room temperature for 1.5 hours. After the reaction was completed, the solvent was evaporated in vacuo and the residue was extracted with EtOAc. The organic layer was dried over MgSO₄ and concentrated in vacuo to afford a desired compound 5 (52 mg, 93% yield) as a white solid.

¹H NMR (CD₃₀D, 500 MHz): δ=7.11 (4H, s), 2.91 (2H, t, J=7.5 Hz), 2.68 (2H, t, J=7.5 Hz), 2.57 (2H, t, J=8.5), 2.28 (1H, dd, J=14.4, 5.7 Hz), 2.07 (1H, dd, J=14.4, 7.1 Hz), 1.91-1.95 (2H, m), 1.57-1.60 (2H, m), 1.20-1.31 (20H, m), 0.95 (3H, d, J=6.6 Hz).

Hereinafter, the compound 5 is referred to as NH₂-BMPP.

### 1-2. Preparation of Fluorescent-Labeled BMPP

1 mg of NH₂-BMPP was dissolved in a solution of 0.8 mL of DMSO and 0.1 mL of Na₂CO₃(10 mM). 50 μL of a solution of Alexa Fluor™ 680-NHS in DMSO (1 mg of Alexa Fluor™ 680-NHS was dissolved in DMSO) was added to the NH₂-BMPP solution so that a final concentration of the Alexa Fluor™ 680-NHS was 0.9 mM. The BMPP was reacted with the Alexa Fluor™ 680-NHS at room temperature for 24 hours. Alexa Fluor™ 680-conjugated BMPP (hereinafter, Alexa Fluor™ 680-BMPP) was purified using size exclusion chromatography (GE-Healthcare, PD10).

### 2. Experimental Example 2: Evaluation of Ability of Leucocytes to Uptake Alexa Fluor™ 680-BMPP Using Whole Blood Collected from Subject

### (I) Method

Each 200 μl of whole blood collected from a healthy individual using a blood collection tube containing heparin was used as a sample. As a fluorescent-labeled BMPP-added group, the Alexa Fluor™ 680-BMPP prepared in the experimental example 1 was added to the samples so that a final concentration of the Alexa Fluor™ 680-BMPP was 0.0074 μM, 0.022 μM, 0.067 μM, and 0.2 μM, respectively. As a negative control group, Alexa Fluor™ 680 NHS Ester hydrolysate (hereinafter, simply referred to as Alexa Fluor™ 680) was added to the samples so that a final concentration of the Alexa Fluor™ 680 was 0.0074 μM, 0.022 μM, 0.067 μM, and 0.2 μM, respectively. Then, these samples were allowed to stand at room temperature for 30 minutes to uptake the fluorescent-labeled substance.

After the uptake is completed, 2 ml of BD FACS Lysing Solution (BD Biosciences) was added to the sample, and the mixture was allowed to stand at room temperature for 30 minutes to hemolyze erythrocytes, and then leucocytes were collected as precipitates by centrifugation at room temperature at 1500 rpm for 30 minutes, and resuspended in PBS to prepare a sample for FACS analysis. The FACS analysis was performed by preparing gates containing lymphocytes, monocytes, or neutrophils in advance by dot plots of forward-scattered light and side-scattered light using leucocytes prepared in the same manner without adding the Alexa Fluor™ 680-BMPP, and measuring the number of cells until 10 million cells were counted in the gate.

### (II) Results

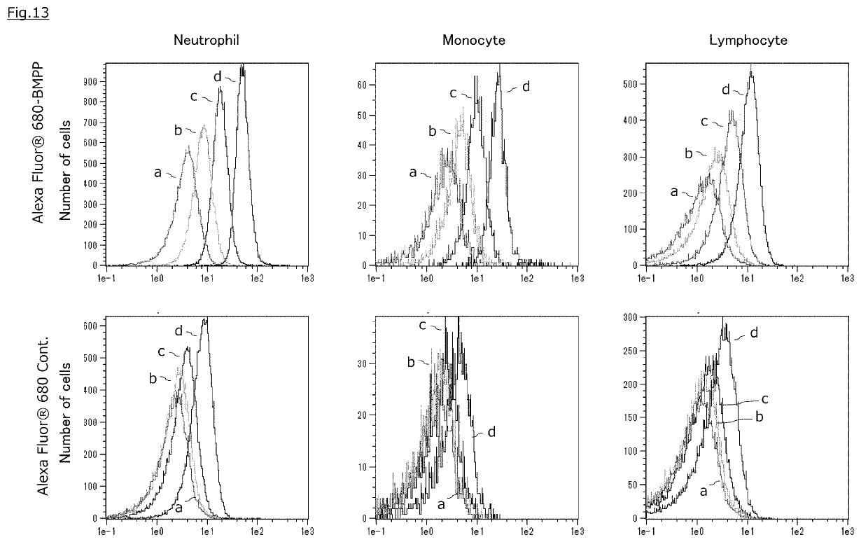

In FIG. 13, the upper part shows the result when the Alexa Fluor™ 680-BMPP is added, and the lower part shows the result when the Alexa Fluor™ 680 is added. Reference signs a, b, c, and d indicate results when the final concentrations are 0.0074 μM, 0.022 μM, 0.067 μM, and 0.2 μM, respectively. The horizontal axis represents a fluorescence intensity of the Alexa Fluor™ 680, and the vertical axis represents the number of cells in the fluorescence intensity.

In all of neutrophils, monocytes, and lymphocytes, the histograms shifted to the rightward in a concentration dependent manner in the Alexa Fluor™ 680-BMPP group in the upper part (that is, the cell population shifted to the higher fluorescence intensity), and thus the concentration dependent increase of uptake amount of the Alexa Fluor™ 680-BMPP was confirmed.

### 3. Experimental Example 3: Evaluation of Ability of Leucocytes to Uptake Fluorescein-Labeled BMPP Using Whole Blood Collected from Subject

### (I) Method

In order to confirm that the uptake of the Alexa Fluor™ 680-BMPP does not depend on a fluorescent dye, fluorescence-labeled BMPP (hereinafter, fluorescein-labeled BMPP) was prepared using fluorescein instead of the Alexa Fluor™ 680. Fluorescein-labeled BMPP was added to the whole blood collected from the healthy individual using the blood collection tube containing heparin so that the final concentration of the fluorescein-labeled BMPP is 0 μM, 0.13 μM, 0.27 μM, or 0.53 μM, and abilities of neutrophils, monocytes, and lymphocytes to uptake the fluorescein-labeled BMPP were evaluated in the same manner as described in experimental example 2.

### (II) Results

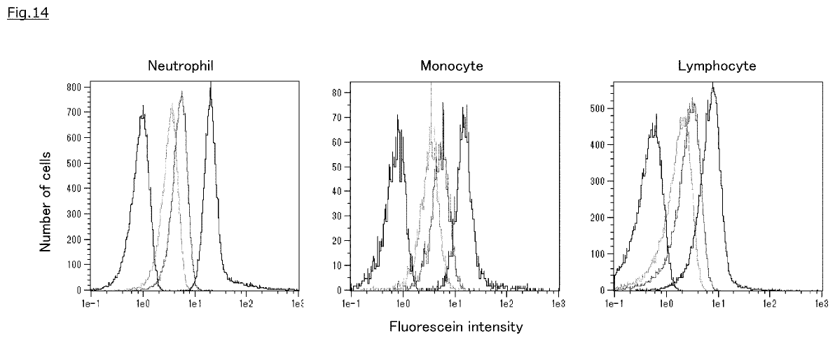

The results are shown in FIG. 14. The curves of each histogram show the final concentrations of 0 μM, 0.13 μM, 0.27 μM, and 0.53 μM from the left. The horizontal axis represents the fluorescence intensity of fluorescein, and the vertical axis represents the number of cells in the fluorescence intensity. Even when BMPP labeled with the fluorescein was used, the concentration dependent increase of uptake amount of the fluorescein-labeled BMPP was confirmed as in the case of using the BMPP labeled with Alexa Fluor™ 680.

### 4. Experimental Example 4: Comparison of Abilities of Leucocytes to Metabolize Fluorescence-Labeled BMPP in Healthy Individuals and Patients with Primary Triglyceride Deposit Cardiomyovasculopathy

Next, the ability of the leucocytes, which is prepared from whole blood of healthy individuals and patients with primary triglyceride deposit cardiomyovasculopathy with an erythrocyte removal reagent, to uptake and excrete the Alexa Fluor™ 680-BMPP was evaluated.

### (I) Method

### (i) Preparation of Leucocytes

5.5 ml of whole blood collected from healthy individuals and patients using the blood collection tube containing heparin was added with the leucocyte separating agent (HetaSep, Veritas Corporation) in an amount of one fifth of the whole blood, leucocytes were collected according to a manufacturer's instruction, and the cells were resuspended in 2.1 ml of DMEM medium without serum (hereinafter, DMEM serum-free medium). Alexa Fluor™ 680-BMPP uptake ability and Alexa Fluor™ 680-BMPP excretion ability of the leucocytes were measured using 400 μl each as described below.

### (ii) Measurement of Alexa Fluor™ 680-BMPP Uptake Ability and Alexa Fluor™ 680-BMPP Excretion Ability Using Leucocytes

40 μl of the Alexa Fluor™ 680-BMPP or 40 μl of the Alexa Fluor™ 680 was added to 400 μl of the cell resuspension so that a final concentration of the Alexa Fluor™ 680-BMPP or the Alexa Fluor™ 680 was 0.067 μM, and a reaction for uptake the fluorescence-labeled substance was performed by standing at room temperature for 30 minutes. A sample for measurement immediately after the uptake was added with BD FACS Lysing Solution immediately after the reaction to be hemolyzed and then, subjected to FACS. On the other hand, as for a sample for measurement of excretion, the Alexa Fluor™ 680-BMPP or the Alexa Fluor™ 680 was removed from the sample by centrifugation at room temperature, cells were resuspended in 500 μl of DMEM serum-free medium, and the cells were allowed to stand at 37° C. for 2 hours to be subjected to an excretion reaction of the taken Alexa Fluor™ 680-BMPP or Alexa Fluor™ 680, then BD FACS Lysing Solution was added, and the sample was subjected to FACS in the same manner as the sample immediately after the uptake. FACS analysis was performed in the same manner as described in the experimental example 2, and for each of neutrophils, monocytes, and lymphocytes, fluorescence intensity of the sample immediately after uptake was compared with fluorescence intensity of the sample for measurement of excretion.

### (II) Results

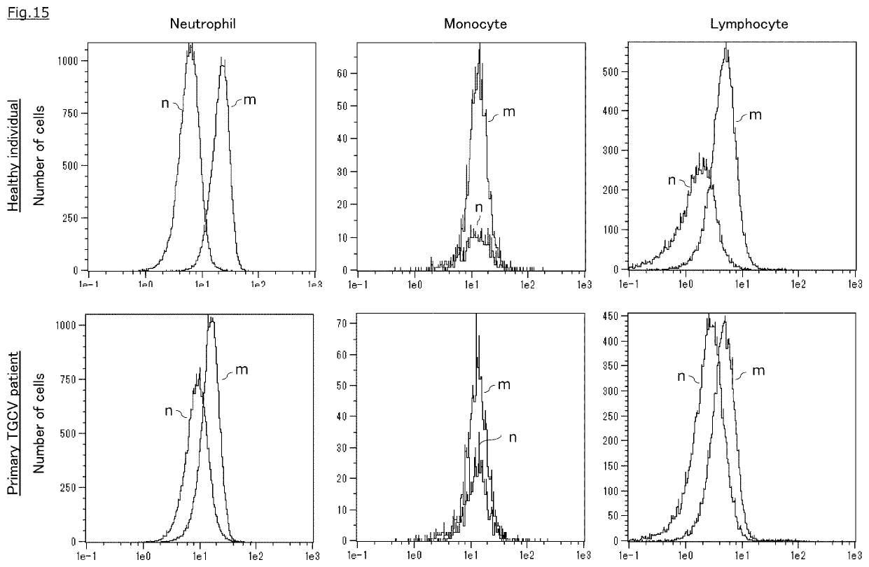

The results are shown in FIG. 15. Reference sign m represents a histogram immediately after the uptake, and reference sign n represents a histogram after the excretion. The upper part of FIG. 15 shows the abilities of neutrophils, monocytes, and lymphocytes in the healthy individuals to excrete the Alexa Fluor™ 680-BMPP. The lower part of FIG. 15 shows the abilities of neutrophils, monocytes, and lymphocytes in the primary TGCV patients to excrete the Alexa Fluor™ 680-BMPP. The ability of neutrophils in the healthy individuals to excrete the Alexa Fluor™ 680-BMPP was sufficiently demonstrated by the shift of the histogram to the leftward (i.e., the shift of the cell population to the lower fluorescence intensity). In addition, the ability of lymphocytes in the healthy individuals to excrete the Alexa Fluor™ 680-BMPP was demonstrated by the shift of the fluorescence intensity as a broad cell population to the leftward. It was demonstrated that the ability of neutrophils in the primary TGCV patients to excrete the Alexa Fluor™ 680-BMPP was clearly reduced because the shift of the fluorescence intensity to the leftward was significantly smaller than that in the healthy individuals. It was demonstrated that the ability of the lymphocytes in the primary TGCV patients to excrete the Alexa Fluorm 680-BMPP was reduced because the shift of the fluorescence intensity to the leftward tends to be small compared to that in the healthy individuals.

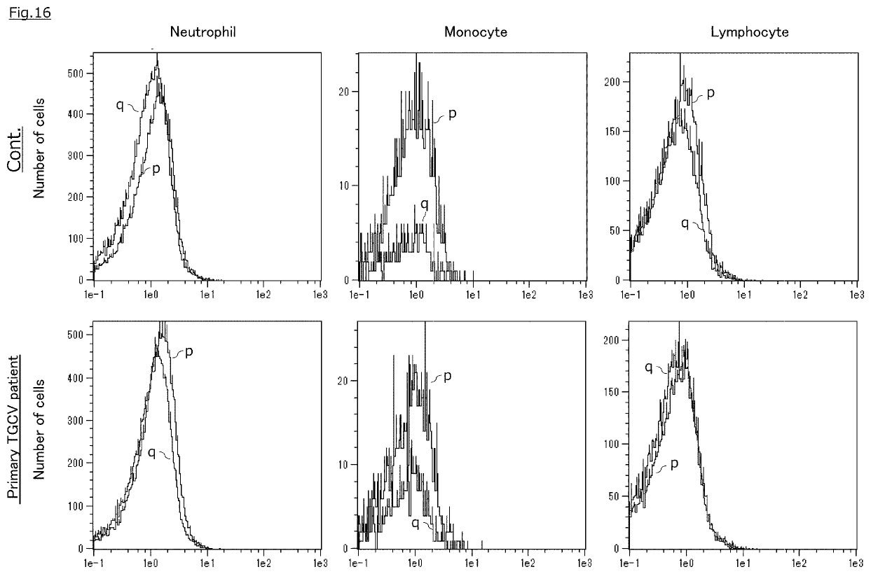

FIG. 16 shows the ability of leucocyte to excrete the Alexa Fluor™ 680. Reference sign p represents a histogram immediately after the uptake, and reference sign q represents a histogram after the excretion. The upper part of FIG. 16 shows the abilities of neutrophils, monocytes, and lymphocytes in the healthy individuals to excrete the Alexa Fluor™ 680. The lower part of FIG. 16 shows the abilities of neutrophils, monocytes, and lymphocytes in the primary TGCV patients to excrete the Alexa Fluor™ 680. There was no difference between the abilities of the leucocytes in the healthy individuals and the leucocytes in the primary TGCV patients to excrete the Alexa Fluor™ 680.

From the above, it was shown that the leucocyte of the primary TGCV patient has a lower ability to excrete the Alexa Fluor™ 680-BMPP than that of the healthy individuals. This result was shown that the lower ability in the primary TGCV patient was not dependent on Alexa Fluor™ 680.

### 5. Experimental Example 5: Examination of Influence on Alexa Fluor™ 680-BMPP Excretion Amount Based on Excretion Time

### (I) Method

### (i) Preparation of Leucocyte Sample

6 ml of each whole blood collected from two healthy individuals using the blood collection tube containing heparin was added with the HetaSep (Veritas Corporation) in an amount of one fifth of the whole blood, leucocytes were collected according to the manufacturer's instruction, and the cells were resuspended in 2.1 ml of the DMEM serum-free medium. Alexa Fluor™ 680-BMPP uptake ability and Alexa Fluor™ 680-BMPP excretion ability of the leucocytes were measured using 400 μl each as described below.

### (ii) Reaction for Uptake of Alexa Fluor™ 680-BMPP

40 μl of the Alexa Fluor™ 680-BMPP was added to 400 μl of the cell resuspension so that a final concentration of the Alexa Fluor™ 680-BMPP was 0.067 μM, and a reaction for uptake the Alexa Fluor™ 680-BMPP was performed by standing at room temperature for 30 minutes. A sample for measurement immediately after the uptake was added with BD FACS Lysing Solution immediately after the reaction to be hemolyzed and then, subjected to FACS. On the other hand, as for a sample for measurement of excretion, the Alexa Fluor™ 680-BMPP was removed from the sample by centrifugation at room temperature, cells were resuspended in 500 μl of DMEM serum-free medium, and the cells were allowed to stand at 37° C. for 0, 10, 30, 60, and 20 minutes to be subjected to an excretion reaction of the taken Alexa Fluor™ 680-BMPP, then BD FACS Lysing Solution was added, and the sample was subjected to FACS in the same manner as the sample immediately after the uptake. FACS analysis was performed in the same manner as described in the experimental example 2, and for each of neutrophils, monocytes, and lymphocytes, fluorescence intensities at each excretion time were compared each other.

### (II) Results

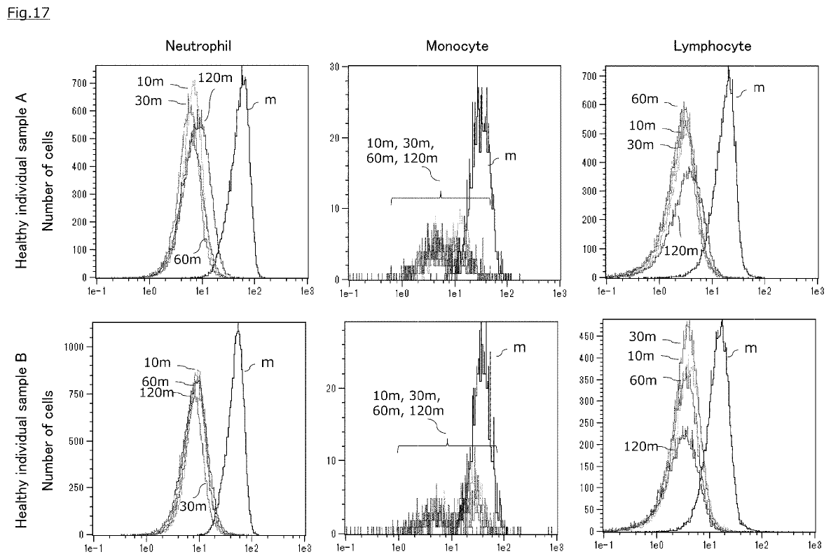

The results are shown in FIG. 17. The upper part of FIG. 17 shows the result of the healthy individual A and the lower part of FIG. 17 shows the result of the healthy individual B. In both healthy individuals, histograms did not shift from the excretion time of 10 minutes to 120 minutes, and it was considered that the taken Alexa Fluor™ 680-BMPP was sufficiently excreted at the time of 10 minutes and almost the background was reached. This suggests that evaluation is possible even with shorter excretion time.

### 6. Reference Example

### 6-1. Reference Example 1: Effect of Medium Chain Fatty Acid

6-week-old (160 to 180 g) male SD rats were used. 18 animals were prepared as a control group, and 8 animals were prepared as a medium chain fatty acid administration group. All rats were subjected to a preliminary-feeding for 1 week after receiving with a normal diet alone, and then the medium chain fatty acid administration group was fed with the normal diet and forcedly administered with 1145 mg/kg body weight/day of high-content triglyceride (98% of constituent fatty acids are decanoic acid). The control group was also fed with the normal diet and forcedly administered with 1145 mg/kg body weight/day of water.

After the lapse of 2 weeks after receiving, all the rats were fed a high-fat diet. In addition, the medium chain fatty acid administration group was forcedly administered with 1145 mg/kg body weight/day of high-content triglyceride. The control group was also forcedly administered with 1145 mg/kg body weight/day of water.

After the lapse of 3 weeks after receiving, abdominal aortic aneurysm induction treatment was performed. The abdominal aortic aneurysm induction treatment is a treatment of inserting a catheter into an abdominal aorta of a rat and ligating the abdominal aorta together with the inserted catheter.

During 4 weeks thereafter, the control group was continued to be fed the high-fat diet and continued to be forcedly administered with 1145 mg/kg body weight/day of water. On the other hand, the medium chain fatty acid administration group was continued to be fed the high-fat diet and continued to be forcedly administered with high-content triglyceride. Then, after the lapse of 4 weeks after the abdominal aortic aneurysm induction treatment was performed, the rats were euthanized and dissected.

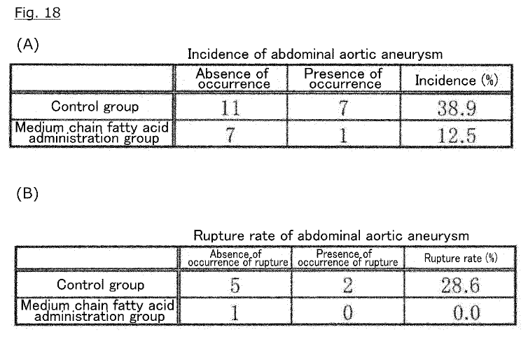

The results are shown in FIG. 18(A). In 1 animal belonging to the medium chain fatty acid administration group, occurrence of abdominal aortic aneurysm was observed.

In 7 animals belonging to the medium chain fatty acid administration group, occurrence of abdominal aortic aneurysm was not observed. From the above, the incidence of abdominal aortic aneurysm was 38.9% in the control group and 12.5% in the medium chain fatty acid administration group.

FIG. 18(B) shows the results of examining the presence or absence of occurrence of the rupture of aortic aneurysm in rats in which the abdominal aortic aneurysms occurred. Referring to a table 2, the rupture of the abdominal aortic aneurysm was observed in 2 animals out of 7 animals belonging to control group. In the remaining 5 animals of the control groups, the rupture of the abdominal aortic aneurysm was not observed.

On the other hand, in 1 animal belonging to the medium chain fatty acid administration group in which the aortic aneurysm occurred, the rupture of the abdominal aortic aneurysm was not observed.

From the above, the incidence of the rupture of the abdominal aortic aneurysm was 28.6% in the control group, and 0.0% in the medium chain fatty acid administration group.

### 6-2. Reference Example 2: Effect of Administration of Tricaprin

### <Case 1>

### (i) Patient

A 44 years old female with genetic ATGL (Adipose triglyceride lipase) deficiency. Since it was confirmed that the patient was suffering from triglyceride deposit-type atherosclerosis by coronary CT inspection, MCT dietary therapy was started. The patient ingested 6 to 9 g of tricaprin as the MCT per 1 day, 3 times a day after meals for 50 days.

### (ii) Evaluation of Fat Accumulated in Coronary Arteries

Before the start of the MCT dietary therapy and after 50 days from the start of the MCT dietary therapy, normal coronary artery CT was performed to acquire a three-dimensional DICOM image of the left anterior descending branch. The samples were resampled in 0.1 mm³ of voxels and layer-by-layer coloring was performed based on intra-voxel CT values. Smoothing was performed between voxels. The layer-by-layer coloring was performed as follows.

Light gray: between −25 HU to 0 HU (fat content is high)
Middle gray 0 HU to 40 HU (fat content is medium)
Dark gray: 40 HU to 125 HU (no fat)
(iii) Results

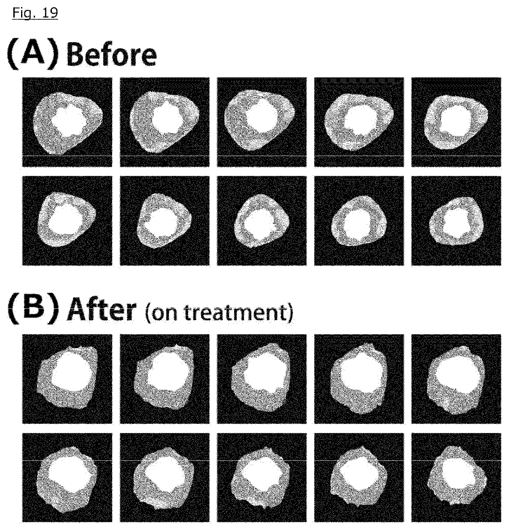

FIG. 19 shows cross-sectional views of the coronary artery in which the layer-by-layer coloring was performed. FIG. 19(A) shows results before the start of the MCT dietary therapy, and FIG. 19(B) shows results after 50 days from the start of the MCT dietary therapy. These results shows that the 50-day MCT diet resulted in regression of the triglyceride deposit-type atherosclerosis of the coronary artery.

### <Case 2>

### (i) Patient

A 65 years old male who has suffered from refractory angina pectoris for years. Even when a combination treatment of a plurality of antianginal drugs and statins or the like was performed, the angina pectoris was rather exacerbated. Since it was confirmed that the patient was suffering from triglyceride deposit-type atherosclerosis by coronary CT inspection, MCT dietary therapy was started. The patient ingested 1.5 g of tricaprin as the MCT per 1 day, 3 times a day after meals for 4 years.

### (ii) Evaluation of Fat Accumulated in Coronary Arteries

Before the start of the MCT dietary therapy and after 4 years from the start of the MCT dietary therapy, normal coronary artery CT was performed, and the layer-by-layer coloring of the inside of the left anterior descending branch was performed in the same manner as described in the case 1.

(iii) Results

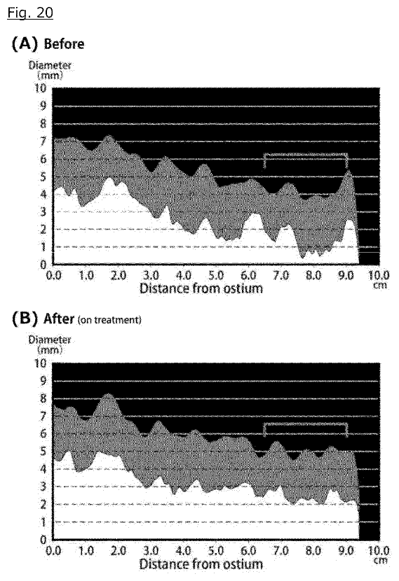

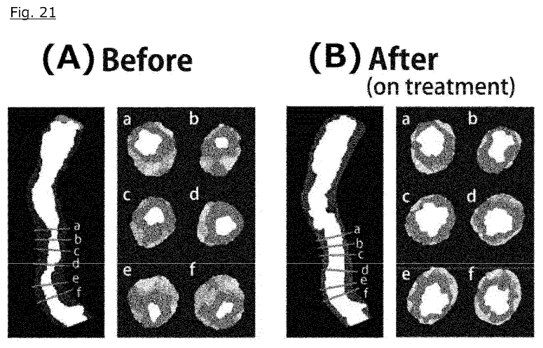

The results are shown in FIGS. 20 and 21. In FIG. 20, the horizontal axis represents the distance from the coronary artery ostium, the vertical axis represents the diameter of the blood vessel, FIG. 20(A) shows the result before the start of the MCT dietary therapy, and FIG. 20(B) shows the result after 4 years from the start of the MCT dietary therapy. White is the blood flow site, and gray is the triglyceride layer. FIG. 21 shows cross-sectional views the blood vessel at 6 positions (a to f) in a portion wherein the distance from the coronary artery ostium to the portion is 6.5 to 9.0 cm. It is clearly shown in FIGS. 20 and 21 that the 4-year MCT diet therapy resulted in regression of the triglyceride deposit-type atherosclerosis of the coronary artery, and the diameter of the blood flow site was significantly increased.

### 7. Experimental Example 6: Comparison of Ability of Leucocytes to Metabolize Fluorescence-Labeled BMPP Before and After Tricaprin Treatment

Next, the abilities for taking up and for excreting the Alexa Fluor™ 680-BMPP were compared using whole blood collected from the patients in the case 1 described in the above 6-2. before and after the tricaprin treatment.

### (i) Method

Preparation of the leucocyte and measurement of the abilities for taking up and for excreting the Alexa Fluor™ 680-BMPP using the leucocyte cells were performed according to the experimental examples.

### (ii) Results

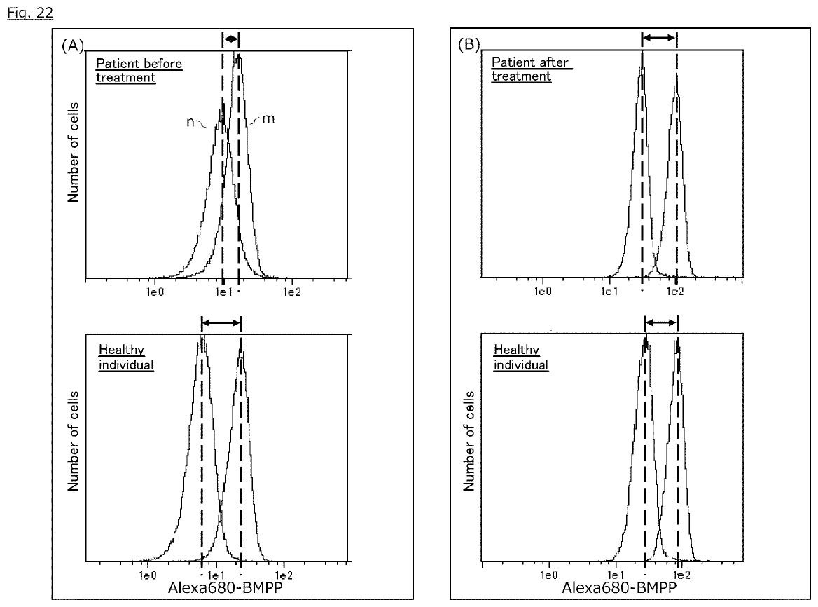

The results are shown in FIG. 22. Reference sign m represents a histogram immediately after the uptake, and reference sign n represents a histogram after the excretion. The upper part of FIG. 22(A) shows the abilities of leucocytes (neutrophils) in the patient before the start of the treatment to excrete the Alexa Fluor™ 680-BMPP. The lower part of FIG. 22(A) shows the abilities of leucocytes in the healthy individual to excrete the Alexa Fluor™ 680-BMPP. The upper part of FIG. 22(B) shows the abilities of leucocytes in the patient after the treatment to excrete the Alexa Fluor™ 680-BMPP. The lower part of FIG. 22(B) shows the abilities of leucocytes in the healthy individual to excrete the Alexa Fluor™ 680-BMPP. As shown in FIG. 22(A), the ability for excreting the Alexa Fluor™ 680-BMPP in the patients before the start of the treatment was clearly reduced because the shift of fluorescence intensity to the left was significantly smaller than that in healthy individuals. As shown in FIG. 22(B), the ability for excreting the Alexa Fluor™ 680-BMPP in the patients after the treatment was restored to the same level as in healthy individuals. These results shows that the ability for excreting the Alexa Fluor™ 680-BMPP is also useful for evaluating the effect of the MCT dietary therapy.

1. A method for acquiring a value relating to a triglyceride metabolic capacity in leucocytes of a subject, comprising following steps of:
a first step of mixing, in vitro, a fatty acid compound labeled with a fluorescent substance with the leucocytes collected from the subject and thus bringing the fatty acid labeled with the fluorescent substance into contact with the leucocytes, wherein the fatty acid compound contains a fatty acid residue, the fatty acid residue has 8 to 26 carbon atoms, and a part of hydrogen atoms constituting the fatty acid residue, excluding a terminal methyl group of the fatty acid residue, may be substituted by an alkyl group having 1 to 3 carbon atoms; and
a second step of acquiring, as the value relating to the triglyceride metabolic capacity, a measured fluorescence intensity that is derived from the fluorescent label in the leucocytes.

2. The method according to claim 1, wherein a hydrogen atom of the terminal methyl group of the fatty acid residue is substituted with a substituted or unsubstituted phenyl group.

3. The method according to claim 1, wherein the fatty acid compound is represented by the following general formula 1: 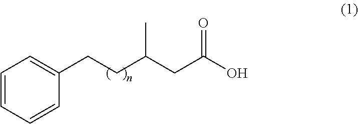
(n is an integer of 4 to 22).

4. The method according to claim 1, wherein the fatty acid compound labeled with the fluorescent substance is represented by the following general formula 2: 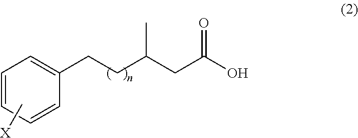
(n is the integer of 4 to 22. X includes the fluorescent substance and a linker moiety).

5. The method according to claim 1, further comprising
a third step of removing the fluorescence-labeled fatty acid compound from the leucocytes over a period of 5 minutes to 2 hours after the second step;
a fourth step of acquiring a measured fluorescence intensity that is derived from the fluorescent label in the leucocytes after the third step; and
a fifth step of obtaining a value indicating a difference between a first measurement value and a second measurement value using the measured fluorescence intensity acquired in the second step as the first measurement value and the measured fluorescence intensity acquired in the fourth step as the second measurement value, and acquiring the value indicating the difference as the value relating to the triglyceride metabolic capacity.

6. A method for presenting disease information, comprising
comparing the value relating to the triglyceride metabolic capacity in the leucocytes of the subject acquired by the method for acquiring the value according to claim 1 with a predetermined reference range of the triglyceride metabolic capacity, and suggesting that the subject is triglyceride deposit cardiomyovasculopathy, triglyceride deposit-type atherosclerosis, or a preliminary group of these diseases when the triglyceride metabolic capacity in the leucocytes of the subject is out of the reference range.

7. A method for presenting disease differentiation information, comprising
comparing the value relating to the triglyceride metabolic capacity in the leucocytes of the subject acquired by the method for acquiring the value according to claim 1 with the predetermined reference range of the triglyceride metabolic capacity, and suggesting that the subject is cholesterol deposit-type atherosclerosis or a preliminary group thereof when the triglyceride metabolic capacity in the leucocytes of the subject is within the reference range.

8. A method for presenting disease differentiation information, comprising
comparing the value relating to the triglyceride metabolic capacity in the leucocytes of the subject acquired by the method for acquiring the value according to claim 1 with the predetermined reference range of the triglyceride metabolic capacity, and suggesting that the subject is not dilated cardiomyopathy when the triglyceride metabolic capacity in the leucocytes of the subject is out of the reference range.

9. A method for presenting efficacy information on a vasodilation therapy using a stent, comprising
comparing the value relating to the triglyceride metabolic capacity in the leucocytes of the subject acquired by the method for acquiring the value according to claim 1 with the predetermined reference range of the triglyceride metabolic capacity, and suggesting that the vasodilation therapy using the stent is not effective in the subject when the triglyceride metabolic capacity in the leucocytes of the subject is out of the reference range.

10. A method for presenting efficacy information of a composition, comprising
comparing the value relating to the triglyceride metabolic capacity in the leucocytes of the subject acquired by the method for acquiring the value according to claim 1 with the predetermined reference range of the triglyceride metabolic capacity, and suggesting that administration of a medium chain fatty acid composition and/or administration of a composition for regression of the triglyceride deposit-type atherosclerosis to the subject is effective when the triglyceride metabolic capacity in the leucocytes of the subject is out of the reference range.

11. A method for presenting therapeutic effect information, comprising
comparing the value relating to the triglyceride metabolic capacity in leucocytes of the subject acquired by the method for acquiring the value according to claim 1 with the value relating to the triglyceride metabolic capacity of the same subject in the past, and suggesting that a therapy applied to the subject is effective when the value relating to the triglyceride metabolic capacity in the leucocytes of the subject is higher than the value relating to the triglyceride metabolic capacity of the same subject in the past.

12. A computing device comprising a processing unit that executes each of the steps according to claim 8.

13. (canceled)

14. A blood collection tube used for blood collection for collecting leucocytes of a subject, wherein
the leucocytes are used to acquire the value relating to the triglyceride metabolic capacity according to claim 1 in vitro, and
the blood collection tube contains a specific gravity liquid for separating the leucocytes or a specific gravity liquid for separating the leucocytes and a leucocyte separating agent.

15. A test reagent for evaluating a triglyceride metabolic capacity, comprising a fatty acid compound labeled with a fluorescent substance, wherein the fatty acid compound contains a fatty acid residue, the fatty acid residue has 8 to 26 carbon atoms, and a part of a hydrogen atoms constituting the fatty acid residue, excluding a terminal methyl group of the fatty acid residue, may be substituted with an alkyl group having 1 to 3 carbon atoms.

16. A test kit comprising the blood collection tube according to claim 14.

17-20. (canceled)
